(19) United States
(12) Patent Application Publication
(10) Pub. No.: US 2024/0341250 A1
(43) Pub. Date: Oct. 17, 2024

(54) METHOD OF ENHANCING THE ORTHONASAL PROFILE OF A PLANT

(71) Applicant: Nicoventures Trading Limited, London (GB)

(72) Inventors: Maria MONTSERRAT SANCHEZ PENA, London (GB); Martine GELINAS, Montreal (CA); Carla RENTON, Cape Town (ZA)

(21) Appl. No.: 18/579,686

(22) PCT Filed: Jul. 14, 2022
(86) PCT No.: PCT/GB2022/051827

§ 371 Date: Jan. 16, 2024

(30) Foreign Application Priority Data
Jul. 16, 2021 (GB) 2110266.0

(51) Int. Cl. A01H 3/02; A24B 15/16; A24B 15/30; G01N 33/00; G16C 20/30; G16C 20/64

(52) U.S. Cl. CPC A01H 3/02; A24B 15/16; A24B 15/303; G01N 33/0098; G16C 20/30; G16C 20/64

(57) ABSTRACT

The present disclosure relates to a method of enhancing the orthonasal profile of a plant, as well as a method of preparing a composition for a substance delivery system, the composition having a predetermined orthonasal profile. The present disclosure further relates to a consumable for use in a substance delivery system, the consumable comprising the composition with the predetermined orthonasal profile.

### TECHNICAL FIELD

The present disclosure relates to a method of enhancing the orthonasal profile of a plant, as well as a method of preparing a composition for a substance delivery system, the composition having a predetermined orthonasal profile. The present disclosure further relates to a consumable for use in a substance delivery system, the consumable comprising the composition with the predetermined orthonasal profile.

### BACKGROUND

Flavoured compositions are used in various substance delivery systems to enhance a user's experience of the substance being delivered. Aerosol provision systems, for example e-cigarettes, generally contain an aerosolisable material such as a reservoir of a source liquid, typically with a flavouring agent and an optional active ingredient, from which an aerosol is generated for inhalation by a user, e.g. by vaporisation. Other source materials may be similarly aerosolised in heating devices, such as a gel or solid material comprising an active ingredient and/or a flavouring agent or flavourant. Flavour can also be introduced in smoking articles such as cigarettes either via the filter element attached to one end of the tobacco rod or via the tobacco material directly. In aerosol-free delivery systems, flavour may be delivered in the form of a pouched oral product such as a smokeless tobacco product, or an oral product containing a tobacco-free substrate.

Irrespective of the product type, however, it is important for flavour to be delivered to the user in an enjoyable form and over a wide spectrum of profiles. In particular, it is desirable if the flavour delivered can be tailored to consumer preference and/or other requirements including any regulatory limitations. For example, it may be preferable to formulate a flavour so as to produce a sensory experience with a particular taste and/or aroma profile.

### SUMMARY

In a first aspect, the present disclosure provides a method of enhancing the orthonasal profile of a Lamiaceae, Geraniaceae, Asteraceae, Myrtaceae or Ericaceae plant, the method comprising:
(a) determining an initial orthonasal profile of the Lamiaceae, Geraniaceae, Asteraceae, Myrtaceae or Ericaceae plant, wherein the profile comprises a plurality of chemical compounds and their respective contents;
(b) modifying the Lamiaceae, Geraniaceae, Asteraceae, Myrtaceae or Ericaceae plant through its growing conditions by controlling one or more parameters selected from light exposure time, temperature, relative humidity, elevation, light intensity, total sun radiation, growth duration or a combination thereof;
(c) determining the orthonasal profile of the modified plant; and
(d) optionally altering one or more parameters of the growing conditions in step (b) to increase and/or reduce the respective content(s) of one or more chemical compound(s) in the modified orthonasal profile relative to the initial orthonasal profile.

In a second aspect, the present disclosure provides a method of preparing a mint-flavoured composition for a substance delivery system, said mint-flavoured composition having a predetermined orthonasal profile with at least one aroma other than mint, the method comprising:
(a) identifying the predetermined orthonasal profile with at least one aroma other than mint, the orthonasal profile comprising a plurality of chemical compounds and their respective contents; and
(b) providing each of the plurality of chemical compound(s) at their respective content(s) by including one or more natural or synthetic source(s) of said compound(s) in the composition.

In a third aspect, the present disclosure provides a mint-flavoured composition obtainable from the method described herein of the second aspect.

In a fourth aspect, the present disclosure provides a consumable for use in a substance delivery system comprising the mint-flavoured composition as described herein of the third aspect, and at least one component selected from aerosol-former material, active substances, substrate materials, or a combination thereof.

In a fifth aspect, the present disclosures provides a container or package containing the consumable described herein of the fourth aspect.

In a sixth aspect, the present disclosure provides a substance delivery system comprising the consumable described herein of the fourth aspect or the container or package described herein of the fifth aspect.

In a seventh aspect, the present disclosure provides a Lamiaceae, Geraniaceae, Asteraceae, Myrtaceae or Ericaceae plant, which has been modified to increase and/or reduce the respective content of one or more chemical compound(s) of its orthonasal profile in comparison to an unmodified plant, wherein the modification is through the growing conditions of the plant, and the one or more chemical compound(s) is associated with an aroma other than mint.

These and other features, aspects, and advantages of the disclosure will be apparent from a reading of the following detailed description together with the accompanying drawings, which are briefly described below. The invention includes any combination of two, three, four, or more of the above-noted features as well as combinations of any two, three, four, or more features or elements set forth in this disclosure, regardless of whether such features or elements are expressly combined in a specific embodiment description herein. This disclosure is intended to be read holistically such that any separable features or elements of the disclosure, in any of its various aspects and embodiments, should be viewed as intended to be combinable unless the context clearly dictates otherwise.

For ease of reference, these and further aspects of the present disclosure are now discussed under appropriate section headings. However, the teachings under each section are not limited to the section in which they are presented; the teachings should be read in the context of the disclosure as a whole.

### BRIEF DESCRIPTION OF THE DRAWINGS

Having thus described aspects of the disclosure in the foregoing general terms, reference will now be made to the accompanying drawings, which are not necessarily drawn to scale. The drawings are exemplary only, and should not be construed as limiting the disclosure.

FIG. 1 is a schematic representation of an electronic aerosol delivery system.

FIG. 2 is a cross-sectional view of a pouched product embodiment, taken across the width of the product, showing an outer pouch filled with a composition of the present disclosure.

FIG. 3 is a comparison of orthonasal profiles obtained according to Example 1 described herein. The identified chemical compounds are along the x axis and their respective content in ng/g are along the y axis.

FIGS. 4 and 5 are comparisons of orthonasal profiles obtained according to Example 3 described herein.

### DETAILED DESCRIPTION

In the following description, a number of specific details are presented in order to provide a thorough understanding of the embodiments of the present disclosure. It is, however, to be understood that this disclosure is not limited to these specific details. It is also to be understood that the terminology employed herein is used for the purpose of describing particular embodiments only and is not intended to be limiting since the scope of the present disclosure will be limited only by the appended claims and equivalents thereof. It will also be apparent that specific details known to the person skilled in the art are omitted for the purposes of clarity where appropriate.

As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water). Reference to “wet weight” refers to the weight of the composition including water or other solvent. Unless otherwise indicated, reference to “weight percent” (or “% by weight”) of a composition reflects the total wet weight of the composition (i.e., including water or other solvent).

In this specification, unless otherwise stated, the term “about” modifying the quantity of an ingredient refers to variation in the numerical quantity that can occur, for example, through typical measuring and liquid handling procedures used for making concentrates or solutions in the real world; through inadvertent error in these procedures; through differences in the manufacture, source, or purity of the ingredients employed, or to carry out the methods; and the like. The term “about” also encompasses amounts that differ due to different equilibrium conditions for a composition resulting from a particular initial mixture. Whether or not modified by the term “about”, the claims include equivalents to the quantities.

The ranges provided herein provide exemplary amounts of each of the components. Each of these ranges may be taken alone or combined with one or more other component ranges.

### Orthonasal Profile

The present disclosure relates firstly to a method of enhancing the orthonasal profile of a plant. The above-mentioned first aspect focuses on enhancing the orthonasal profile of a Lamiaceae, Geraniaceae, Asteraceae, Myrtaceae or Ericaceae plant, but the disclosure is not limited to these plants. The skilled person will appreciate from the disclosure as a whole that the teachings herein can be applied to any plant, the Lamiaceae, Geraniaceae, Asteraceae, Myrtaceae or Ericaceae plants are solely being used as an example. Accordingly the present disclosure provides a method of enhancing the orthonasal profile of a plant, the method comprising:
a. determining an initial orthonasal profile of the plant, wherein the profile comprises a plurality of chemical compounds and their respective contents;
b. modifying the plant through its growing conditions by controlling one or more parameters selected from light exposure time, temperature, relative humidity, elevation, light intensity, total sun radiation, growth duration or a combination thereof;
c. determining the orthonasal profile of the modified plant; and
d. optionally altering one or more parameters of the growing conditions in step (b) to increase and/or reduce the respective content(s) of one or more chemical compound(s) in the modified orthonasal profile relative to the initial orthonasal profile.

In various embodiments of the present disclosure, the method includes step (d).

The plant can be any plant known in the art from which an orthonasal profile may be determined. For example, the plant may be a tobacco plant, a herbaceous plant, a cannabis plant, a flowering plant, a spice plant, hops or the like. The plant may, for example be a member of the Cannabaceae or Nicotiana families, or may be a culinary herb or spice. The latter are a known group of plants in the art and include e.g. anise, basil, bay, cardamom, cayenne pepper, cinnamon, cilantro, cumin, curry leaf, dill, elderflower, fennel, garlic, ginger, jasmine, juniper berry, kaffir lime leaves, lemon balm, lemongrass, marjoram, mint, mustard, nutmeg, oregano, parsley, pepper, rosemary, saffron, tarragon, thyme, turmeric, vanilla, and watercress. The Cannabaceae family include Cannabis, and Humulus (hops). The discussion below in relation to the method with a Lamiaceae, Geraniaceae, Asteraceae, Myrtaceae, or Ericaceae plant applies equally to the more general method above.

The term “enhancing” is used herein to mean customizing or adapting the orthonasal profile of the plant to a desired end result. As noted above, it is important for flavour to be delivered to a user in an enjoyable form and over a wide spectrum of profiles. In particular, it is desirable if the flavour can be tailored to consumer preference and/or other requirements including any regulatory limitations.

Flavour is defined by ASTM International (ASTM International, 2009. Standard Terminology Relating to Sensory Evaluations of Materials and Products, E253-09a; incorporated herein by reference) as:
(1) the perception resulting from stimulating a combination of the taste buds, the olfactory organs, and chemesthetic receptors within the oral cavity; or
(2) the combined effect of taste sensations, aromatics and chemical feeling factors evoked by a substance in the oral cavity.

This definition thus takes into account the three elements of flavour: (1) taste, (2) aromatics or aroma, and (3) trigeminal sensations.

Aromatics or aroma refers to a scent or smell and in the present disclosure refers to the orthonasal smell of an object, i.e. the perception of molecules that enter directly through the nose and up the nasal passages. Orthonasal smell is different from retronasal smell or retronasal olfaction, the latter is the ability to perceive flavour dimensions of food and drinks and is associated with molecules travelling up through the nasal passages as one is chewing.

The present disclosure is only concerned with orthonasal smell, not retronasal smell, and the method of the first aspect enables the person skilled in the art to adapt the orthonasal profile of a plant, e.g. a Lamiaceae, Geraniaceae, Asteraceae, Myrtaceae or Ericaceae plant in order to enhance certain aromas thereof.

By way of example, existing plants from the Lamiaceae family such as plants from the Mentha genus, have an orthonasal profile dominated by mint aroma compounds. Mentha suaveolens Variegata or ‘Pineapple mint’, for instance, has an orthonasal profile dominated by e.g. piperitone oxide, menthone, alpha-pinene, beta-pinene and 1,8-cineole, all contributing to the ‘mint’ aroma of the plant. A ‘mint’ aroma may be described as “fresh”, “cool”, “cold”, “refreshing” and/or “clean”. It would be readily identified by the person skilled in the art.

The dominating ‘mint’ aroma compounds mean that to most consumers, ‘pineapple mint’ has an orthonasal profile which is substantially indistinguishable from ‘mint’. In other words, the overriding aroma from the Mentha suaveolens Variegata plant is ‘mint’, and not ‘pineapple’ or any other ‘fruity’ aroma. There is, however, a demand for more variety in the flavour space especially in the mint flavour space, and it would therefore be useful if such Lamiaceae plants (or equally Geraniaceae, Asteraceae, Myrtaceae, and/or Ericaceae plants) could be grown to purposively enhance and/or reduce one or more compounds in its orthonasal profile. For example, in the context of ‘Pineapple mint’, it would be desirable if those compounds responsible for the aroma(s) other than mint—e.g. the orthonasal compounds responsible for the ‘fruity’ aroma—could be encouraged and thereby increased in concentration during growth of the plant so that a consumer of the plant, e.g. in a substance delivery system as described herein, would experience both a mint and a fruity aroma from a consumable containing the plant or extract thereof.

A similar challenge arises for e.g. other ‘fruity’ mints including apple mint (Mentha suaveolens) and strawberry mint (Mentha spicata subsp. citrata), as well as ‘citrus’ mints, e.g. orange mint (Mentha x piperita f. citrata ‘Orange’) and lemon mint (Mentha x piperita f. citrata ‘Lemon’, and other mint varieties having an aroma other than mint, e.g. chocolate mint (Mentha x piperita ‘Chocolate’). The challenge is of course not limited to mint plants; they are just being used as an example for the purposes of the present disclosure.

The method of the first aspect thus enables the person skilled in the art to favour one or more aromas in the orthonasal profile of a plant, and thereby deliver a “tailor-made” or “refined” orthonasal experience to a consumer. Additionally, the method of the first aspect can be used to gather information on the compound(s) that are responsible for one or more aromas in the orthonasal profile of a plant. With this information, it is possible for the person skilled in the art to re-create a particular orthonasal profile via a biomimetic approach. This approach is discussed in more detail in connection with the second aspect, but that discussion is equally applicable to the disclosure of the first aspect. The gathered information may, for example, be used to guide the decisions on which compound(s) to elevate/reduce in the orthonasal profile of the plant and hence the growing conditions controlled in the method herein described.

An ‘orthonasal profile’ of a plant is well-understood in the art; it comprises a plurality of chemical compounds and their respective contents, and is determined using methods known in the art. The orthonasal profile of a plant may, for instance, be determined by headspace technology such that the respective content(s) of the identified compound(s) are headspace concentrations measured in ng per g, otherwise expressed as parts per billion (ppb).

Headspace technology is a technique to elucidate the odour or aroma compounds present in the air surrounding an object of interest, e.g., a part of a plant. The equipment to carry out the technique is readily available and involves a hollow dome or sphere-like object, which forms an airtight seal and surrounds the object of interest. Inert gases are passed into the space containing the object, or a vacuum is established such that the odour or aroma compounds are removed from the headspace. These compounds are in turn captured using a variety of techniques, among them cold surfaces, solvent traps, and adsorbent materials. Once captured, the samples can be analysed using techniques such as gas chromatography, mass spectrometry, carbon-13 NMR, and combinations thereof, and the elucidated compounds compared against known libraries of aroma compounds to identify the scent being produced by the identified compounds.

Laboratory techniques that may be used include solid-phase microextraction, dynamic headspace extraction or stir bar sorption extraction. Typically, solid-phase microextraction or SPME may be used. SPME is a solid phase extraction sampling technique that involves the use of a fibre coated with an extracting phase, that can be a liquid (e.g. polymer) or a solid (sorbent) which extracts different analytes (including both volatile and non-volatile) from different kinds of media, that can be in liquid or gas phase. After extraction, the SPME fibre is transferred to the injection port of separating instruments such as a gas chromatography for analysis. There are many commercially available SPME fibre coatings, and analytical techniques such as gas chromatography are well-known and understood in the art.

In various embodiments of the present disclosure, the orthonasal profile may be determined using headspace technology which comprises gas chromatography (GC) coupled with a secondary technique. The secondary technique may comprise mass spectrometry (MS), olfactometry (O), or a combination thereof. For example, the present disclosure may comprise GC-MS, GC-O or a combination thereof. The combination of GC-MS with GC-O can be useful to identify those compound(s) which are detectable by a human nose. GC-MS with GC-O may therefore be used to provide a threshold orthonasal profile as discussed herein, namely a profile of compounds detectable by a human nose.

In the method of the first aspect, an initial orthonasal profile is determined for the plant. For example, an initial orthonasal profile may be determined for the Lamiaceae, Geraniaceae, Asteraceae, Myrtaceae or Ericaceae plant. The profile may be determined using headspace technology as discussed hereinabove, wherein the concentrations of the identified compounds are specified in ng per g.

In various embodiments of the present disclosure, the headspace of the plant may be characterised using a sample thereof, for example the sample may be a shoot harvested from the plant. A “shoot” in the context of the present disclosure consists of stems and their appendages, e.g. stems, the leaves and lateral buds, flowering stems and flower buds. In various embodiments of the present disclosure, the sample used for headspace analysis may be any part of a plant such as one or more leaves, stems, flowers etc., the present disclosure is not limited in this respect. In various embodiments the part of the plant may comprise a shoot without flowering stems and/or flower buds. In the present disclosure, the orthonasal profile is not determined from an extracted oil of the plant, it is determined from a physical extract of the plant per se.

The method may further comprise comparing the concentrations of the identified compounds with the odour threshold value of the compound to generate a threshold orthonasal profile or orthonasal profile based on odour activity values. The person skilled in the art knows from their common general knowledge where to locate threshold concentrations. These threshold concentrations are known in the art as odour threshold values, odour detection values or aroma threshold values and may be referenced herein by any of these terms. They are the minimal concentration that can be detected by a human nose, and are available from e.g. (Devos, M., Patte, F., Roualt, J., Laffort, P. and Van Gemert, L. J. (1990) Standardized Human Olfactory Thresholds, and The Good Scents Company website: http://www.thegoodscentscompany.com/index.html. Each of these disclosures is incorporated herein by reference.

In various embodiments of the present disclosure, the method of the first aspect thus comprises acquiring data from the plant, wherein the data characterises the headspace of the plant, identifying from the acquired data a plurality of chemical compounds and their respective contents within the headspace, and comparing the plurality of chemical compounds and their respective contents with one or more libraries, the comparison with one or more libraries may be to characterize the plant by one or more aroma types and/or to generate a threshold orthonasal profile. In various embodiments, the comparison with one or more libraries comprise a comparison with an aroma threshold library so as to select the compound(s) and respective content(s) associated with the orthonasal profile of the plant detectable by a human nose. The orthonasal profile thereby characterises the plant by its odour activity values; in other words, the respective content(s) of the chemical compound(s) are odour activity values.

The orthonasal profile of the plant, e.g. the Lamiaceae, Geraniaceae, Asteraceae, Myrtaceae or Ericaceae plant, is determined both at an initial timepoint, for example from a cultivar not subjected to the method of the first aspect, and after modifying the plant through its growing conditions as discussed in more detail below. The determination of the initial orthonasal profile and the orthonasal profile following modification of the plant should be carried out in the same manner, e.g. relying on odour activity values and/or the same analytical technique(s) to ensure that the comparison between the two profiles is meaningful.

Step (a) of the method—determining an initial orthonasal profile of the plant—may also involve consulting a library of predetermined orthonasal profiles. In such embodiments of the present disclosure, the person skilled in the art would therefore rely on the predetermined orthonasal profile as the starting point for the method, rather than data acquired from the plant. The library of profiles may be a result of the method described herein or a pre-existing collection of data from a third party or the like. In various embodiments of the present disclosure, step (a) therefore may comprise identifying a predetermined orthonasal profile for the plant, e.g. a predetermined orthonasal profile for the Lamiaceae, Geraniaceae, Asteraceae, Myrtaceae or Ericaceae plant, wherein the profile comprises a plurality of chemical compounds and their respective contents.

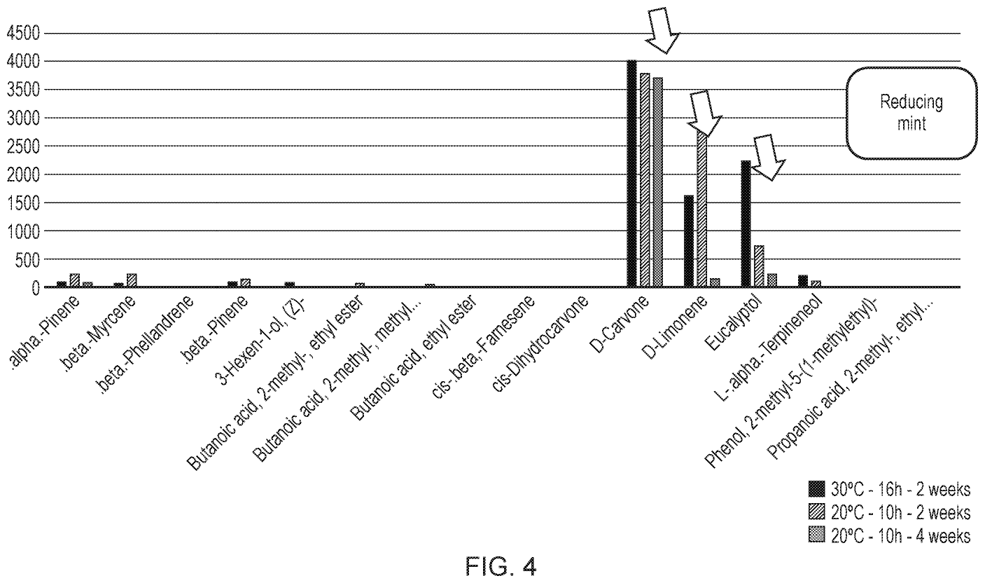

In various embodiments of the present disclosure, and as exemplified by FIG. 3, the method of the first aspect as defined herein may repeat step (c) after step (d). In such embodiments, the method may comprise steps (a), (b), (c) and (d), followed by (c′) determining the orthonasal profile of the plant from step (d). For example:
a. determining an initial orthonasal profile of the plant, wherein the profile comprises a plurality of chemical compounds and their respective contents;
b. modifying the plant through its growing conditions by controlling one or more parameters selected from light exposure time, temperature, relative humidity, elevation, light intensity, total sun radiation, growth duration or a combination thereof;
c. determining the orthonasal profile of the modified plant;
d. altering one or more parameters of the growing conditions in step (b) to increase and/or reduce the respective content(s) of one or more chemical compound(s) in the modified orthonasal profile relative to the initial orthonasal profile; and
c′. determining the orthonasal profile of the plant from step (d).

The method may further comprise comparing the orthonasal profile of (c) with the orthonasal profile of (c′)—as exemplified by FIG. 3. Such a comparison should indicate the effect of the parameter(s) altered in step (d).

The method may further repeat step (d) after step (c′). For example, step (c′) may be followed by step (d′) altering one or more additional or alternative parameters of the growing conditions; such alteration may increase the respective content(s) of one or more chemical compound(s) in the orthonasal profile relative to the initial orthonasal profile of step (a) and/or the orthonasal profile of step (c) and/or the orthonasal profile of step (c′). Step (d′) should be followed by (c″) determining the orthonasal profile of the plant from step (d′), and a comparison can be made between the orthonasal profiles obtained in step (c), (c′) and/or (c″) to identify the effect of the parameter(s) on the orthonasal profile of the plant.

The person skilled in the art will appreciate that the method may further comprise any number of steps (c) and/or (d), to identify and suitable control the parameter(s) of the growing conditions as necessary for the desired orthonasal profile. As noted above, the desired orthonasal profile for the Lamiaceae, Geraniaceae, Asteraceae, Myrtaceae, or Ericaceae plant may be one in which the respective content(s) of one or more chemical compound(s) associated with an aroma other than mint are increased. Optionally the respective content(s) of one or more chemical compound(s) associated with the mint aroma may also be decreased.

In this way, the person skilled in the art is able to tailor the orthonasal profile of the plant according to consumer demand, regulatory demand or otherwise.

### Growing Conditions

The method of the first aspect includes a step of modifying the plant through its growing conditions by controlling one or more parameters selected from light exposure time, temperature, relative humidity, elevation, light intensity, total sun radiation, growth duration or a combination thereof. In various embodiments of the present disclosure, the plant is a Lamiaceae, Geraniaceae, Asteraceae, Myrtaceae or Ericaceae plant.

Each of the above-listed parameters and their measurement methods are known in the art. The temperature refers to a 24 hour period, preferably segmented into a ‘day’ temperature and a ‘night’ temperature. The number of hours within the 24 hour period which are at ‘day’ temperature is not limited and can depend on the light regime being implemented. The light exposure time is associated with the ‘day’ temperature; it is the period of time for which the ‘day’ temperature and light conditions are maintained. The growth duration is the length of time the plant is permitted to grow under the modified growing conditions. Relative humidity is measured using a hygrometer. Elevation is the height of the plant above a fixed reference point, specifically above sea level. Total sun radiation may also be referred to as solar irradiation, solar exposure or solar insolation; it is the power per unit area received from the sun in the form of electromagnetic radiation over a given time period—typically 24 hours or a day. It is used to report the daily radiant energy emitted into the surrounding environment and uses the units Joules/cm²/day.

In various embodiments of the present disclosure, the method comprises controlling one or more parameters selected from light exposure time, temperature, relative humidity, light intensity, total sun radiation, growth duration or a combination thereof. For example, the method may comprise controlling one or more parameters selected from light exposure time, temperature, relative humidity, light intensity, total sun radiation, growth duration or a combination thereof, such as light exposure time, temperature, total sun radiation, growth duration or a combination thereof, particularly light exposure time, temperature and growth duration. The growth duration may be between 1 week and 6 weeks, such as between 1 week and 4 weeks.

In various embodiments of the present disclosure, the growth duration is kept constant (e.g. 4 weeks) and one or more parameters selected from light exposure time, temperature, relative humidity, elevation, light intensity, total sun radiation, or a combination thereof, are controlled. In alternative embodiments of the present disclosure, the growth duration is modified (e.g. between 1 week and 4 weeks) along with one or more parameters selected from light exposure time, temperature, relative humidity, elevation, light intensity, total sun radiation, or a combination thereof.

In various embodiments of the present disclosure, the growth duration is kept constant (e.g. 4 weeks) and one or more parameters selected from light exposure time, temperature or a combination thereof, are controlled. In alternative embodiments of the present disclosure, the growth duration is modified (e.g. between 1 week and 4 weeks) along with one or more parameters selected from light exposure time, temperature, or a combination thereof.

In various embodiments of the present disclosure, the method comprises controlling total sun radiation between 50 and 400 Joules/cm²/day. In various embodiments of the present disclosure, the method comprises controlling total sun radiation between 100 and 400 Joules/cm²/day, such as between 150 and 400 Joules/cm²/day, including 150 and 300 Joules/cm²/day.

In various embodiments of the present disclosure, the method comprises controlling the temperature between 10° C. and 40° C., with the ‘day’ temperature being at least 2° C. higher than the ‘night’ temperature. For example, the ‘day’ temperature may be between 15° C. and 35° C., and the ‘night’ temperature may be between 10° C. and 30° C., provided that the day temperature is at least 2° C. (e.g. at least 5° C.) higher than the night temperature. The time associated with the ‘day’ temperature, namely the light exposure time, may be between 8 and 24 hours, such as between 8 and 20 hours or between 10 and 20 hours.

The person skilled in the art will understand that the exact growing conditions and the control thereof will depend on the particular plant. Some examples are provided in the experimental examples section below. These examples include the following tests:
Test 1: light exposure time of 10 hours—20° C. day—15° C. night
Test 2: light exposure time of 10 hours—30° C. day—25° C. night
Test 3: light exposure time of 16 hours—20° C. day—15° C. night
Test 4: light exposure time of 16 hours—30° C. day—25° C. night

In various embodiments of the present disclosure, the method comprises altering light exposure time and temperature to increase the respective content(s) of one or more chemical compound(s) in the modified orthonasal profile relative to the initial orthonasal profile. The one or more chemical compound(s) may be associated with an aroma other than the dominant aroma of the initial orthonasal profile. By the term “dominant” is meant the main aroma experienced by a human nose, for example the mint aroma from a Mentha plant. In various embodiments of the present disclosure, the method comprises altering light exposure time and temperature to increase the respective content(s) of one or more chemical compound(s) in the modified orthonasal profile relative to the initial orthonasal profile, wherein said chemical compound(s) is associated with an aroma other than mint.

In various embodiments of the present disclosure, the light exposure time is between about 8 hours and about 24 hours. In various embodiments of the present disclosure, the light exposure time is between about 8 hours and about 20 hours. In various embodiments of the present disclosure, the light exposure time is between about 10 hours and about 20 hours, such as 10 hours or 16 hours. In various embodiments of the present disclosure the temperature comprises a day temperature associated with the light exposure time and a night temperature. The day temperature is at least about 2° C., e.g. at least about 5° C., higher than the night temperature. The day temperature may be between about 15° C. and about 35° C., and the ‘night’ temperature may be between about 10° C. and about 30° C. For example, the day temperature may be between about 20° C. and about 35° C., and the ‘night’ temperature may be between about 10° C. and about 25° C., provided that the day and night temperature are different.

### Biomimetic Approach

The method of the second aspect of the present disclosure is intended to capture a biomimetic approach. In particular, an approach which enables a person skilled in the art to identify a predetermined orthonasal profile and to re-create the predetermined orthonasal profile with one or more natural and/or synthetic source(s) of the compound(s) in said profile. For example, a predetermined orthonasal profile may be re-created by combining one or more botanicals or constituent thereof optionally with one or more naturally isolated compounds and/or one or more synthetic compounds.

The biomimetic approach provided by the method of the second aspect thus allows a person skilled in the art to be flexible when re-creating a predetermined orthonasal profile. Such flexibility may be beneficial to meet regulatory demands or consumer preferences. For example, the approach enables an orthonasal profile to be provided by solely natural or synthetic sources and/or a certain type of botanical. It could, for instance, be used to expand the flavour profile of the Lamiaceae, Geraniaceae, Asteraceae, Myrtaceae or Ericaceae botanicals without needing to use synthetic or artificial substances.

As an example, the predetermined orthonasal profile may be delivered solely by one or more botanicals or constituents thereof, the botanical being a member of the Lamiaceae, Geraniaceae, Asteraceae, Myrtaceae or Ericaceae families. In various embodiments of the present disclosure, the predetermined orthonasal profile may be delivered solely by one or more Lamiaceae botanicals or constituents thereof. In various embodiments of the present disclosure, the predetermined orthonasal profile may be delivered solely by one or more Geraniaceae botanicals or constituents thereof. In various embodiments of the present disclosure, the predetermined orthonasal profile may be delivered solely by one or more Asteraceae botanicals or constituents thereof. In various embodiments of the present disclosure, the predetermined orthonasal profile may be delivered solely by one or more Myrtaceae botanicals or constituents thereof. In various embodiments of the present disclosure, the predetermined orthonasal profile may be delivered solely by one or more Ericaceae botanicals or constituents thereof.

Additionally the biomimetic approach enables the person skilled in the art to ensure that compounds included in a consumable for a substance delivery system as described herein are safe for human consumption.

The method of the second aspect is described herein for providing a mint-flavoured composition for a substance delivery system, said mint-flavoured composition having a predetermined orthonasal profile with at least one aroma other than mint. The method is not, however, limited to providing such a mint-flavoured composition. The biomimetic approach expands the flexibility of flavour delivery across the spectrum of available flavours.

Accordingly, the method of the second aspect applies more generally to the provision of a flavoured composition or flavouring agent for a substance delivery system, said composition or agent having a predetermined orthonasal profile with at least a first aroma associated with the primary flavour of the composition and at least a second aroma which is different from the first aroma. The method comprises:
a. identifying a predetermined orthonasal profile with at least a first aroma associated with the primary flavour of the composition and at least a second aroma which is different from the first aroma, the orthonasal profile comprising a plurality of chemical compounds and their respective contents; and
b. providing each of the plurality of chemical compound(s) at their respective content(s) by including one or more natural or synthetic source(s) of said compound(s) in the composition.

In the discussion that follows, the preparation of a mint-flavoured composition will be used as an example of the applicability of the biomimetic approach. As noted above, however, the present disclosure is not limited in this respect. The method of the second aspect applies to any flavoured composition having a first aroma associated with the primary flavour (e.g. mint) and at least a second aroma which is different from the first aroma (e.g. bergamot and orange), the composition being prepared from a predetermined orthonasal profile.

The term “primary flavour” is used interchangeably herein with the term “dominant” or “dominating” flavour. It refers to the flavour that is detected most strongly by a user and thus comprises the majority of the flavour profile of the composition, e.g. greater than 50% of compounds in the flavour profile are responsible for the primary flavour. Typically it will be how the flavour is described. For example, a “mint-flavoured composition” would be characterised herein as having mint as the primary flavour. Similarly, a coffee-flavoured composition would have coffee as the primary flavour.

The “first aroma associated with the primary flavour of the composition” is used herein to refer to the aroma associated with the main flavour of the composition. In the orthonasal profile of the composition, the compounds responsible for the “first aroma” will typically comprise greater than 50% of the compounds in the orthonasal profile. In various embodiments, the orthonasal profile will be the threshold orthonasal profile as described hereinabove. In this respect, the above discussion of the orthonasal profile in the context of the first aspect applies equally to the method of the second aspect. For example, the respective contents of the plurality of chemical compounds may be odour activity values.

The “second aroma which is different from the first aroma” is, in the context of the mint-flavoured composition, also referred to herein as “an aroma in addition to mint”. Essentially it means a second, different smell or aroma to the aroma associated with the primary flavour. The second aroma is not limited and can be any aroma known to the skilled person and detectable by the human nose.

In various embodiments of the present disclosure, the predetermined orthonasal profile may be selected from a library of orthonasal profiles. The profile may be used per se, namely without modification once selected from the library, or it may be altered by selecting one or more portions of the orthonasal profile and/or combining one or more of said portions with at least a portion of a further orthonasal profile. In various embodiments of the present disclosure, the predetermined orthonasal profile may comprise one or more portions of different orthonasal profiles. For example, the predetermined orthonasal profile may comprise two or more portions of different orthonasal profiles.

The “different orthonasal profiles” may relate to different aroma types, such as citrus, fruity, floral, green, herbal, vegetable, creamy, roasted, nutty, woody, spicy or cheesy. The predetermined orthonasal profile may therefore be unique and novel in itself. A flavour team may, for instance, define a desired orthonasal profile for a consumable, which profile is not found in existing libraries but comprises a combination of orthonasal profile portions, e.g. mint with bergamot, orange zest and fruity notes. The method of the present disclosure, with this predetermined orthonasal profile, enables the skilled person to deliver such a unique combination of aromas.

In various embodiments of the present disclosure, the predetermined orthonasal profile may represent a percentage of a profile selected from a library of orthonasal profiles. The percentage may be between about 50% and about 100%, between about 60% and about 100%, between about 70% and about 100%, between about 80% and about 100%.

In various embodiments of the present disclosure, the predetermined orthonasal profile of the claimed method may be less than 100% of a profile selected from a library of orthonasal profiles. As noted above, the predetermined orthonasal profile may therefore be unique and novel in itself. The predetermined orthonasal profile may be between about 50% and about 95% of a profile selected from a library of orthonasal profiles, between about 60% and about 95%, between about 70% and about 95%, or between about 80% and about 95%. For example, a known orthonasal profile may comprise 30 chemical compounds and their respective contents, and the predetermined orthonasal profile used in the claimed method may comprise 20 of the 30 chemical compounds in the known profile.

Instead of or in addition to relying on a library of orthonasal profiles, the predetermined orthonasal profile may be identified by a method which comprises acquiring data from a plant, e.g. a Lamiaceae, Geraniaceae, Asteraceae, Myrtaceae or Ericaceae plant, wherein the data characterises the headspace of the plant; identifying from the acquired data a plurality of chemical compounds and their respective contents within the headspace; comparing the plurality of chemical compounds and their respective contents with one or more libraries to characterize the plant by one or more aroma types; and selecting one or more chemical compound(s) and respective content(s). The latter step of selecting one or more chemical compound(s) and respective content(s) may tie in with the modification discussed above, namely selecting one or more portions of the orthonasal profile and/or combining one or more of said portions with at least a portion of a further orthonasal profile, clearly “selecting one or more portions of the orthonasal profile” encompasses selecting one or more chemical compound(s) and respective content(s).

It will be appreciated by the person skilled in the art that the method of the first aspect may be combined with the method of the second aspect and vice versa. The plant with an enhanced orthonasal profile may, for instance, be analysed to provide the predetermined orthonasal profile or portion thereof for the biomimetic approach disclosed herein. The biomimetic approach and its predetermined orthonasal profile may alternatively be used to make decisions on the enhanced orthonasal profile desired in the method of the first aspect.

In various embodiments of the present disclosure, the method further comprises selecting one or more chemical compound(s) and their respective content(s) which are associated with the second aroma discussed above, and providing one or more of said second aroma compound(s) at its respective content(s) by including a natural or synthetic source of said compound(s) in the composition. The method throughout this disclosure includes modifying the respective content(s) of the selected chemical compound(s), e.g. increasing the content if the compound(s) is associated with the second aroma.

In various embodiments of the present disclosure, the method further comprises selecting one or more chemical compound(s) and optionally their respective content(s) which are associated with the mint aroma, and providing one or more of said mint aroma compound(s) optionally at its respective content(s) by including a natural or synthetic source of said compound(s) in the composition. The method may further comprise selecting one or more chemical compound(s) and optionally their respective content(s) which are associated with the aroma other than mint, and providing one or more of said other aroma compound(s) optionally at its respective content(s) by including a natural or synthetic source of said compound(s) in the composition.

The plurality of chemical compound(s) provided by the one or more natural or synthetic sources thereof are not limited. They are compounds known in the art to be associated with aroma. In various embodiments of the present disclosure, including the method described above for the first aspect, the one or more chemical compound(s) is selected from the group consisting of esters, alcohols, lactones, terpenes, aldehydes or a combination thereof. In various embodiments of the present disclosure, the one or more chemical compound(s) is selected from the group consisting of esters, lactones, terpenes, or a combination thereof.

### Natural or Synthetic Sources

As discussed above, the compound(s) in the orthonasal profile are provided by one or more natural or synthetic source. Any natural or synthetic source may be used which is known to the person skilled in the art, provided that if all compounds in the predetermined orthonasal profile are provided by a natural source, each natural source is not an oil extracted from a botanical.

Any combination of natural and/or synthetic sources may be used to provide the predetermined orthonasal profile and the discussion below is merely provided as an exemplary embodiment, the present disclosure is not limited in this respect.

In various embodiments of the present disclosure, one or more chemical compound(s) in the predetermined orthonasal profile may be provided by a natural source. The natural source is not limited and can include any known natural source of the desired compound, subject to the above-mentioned proviso. The compound may, for instance, be naturally occurring.

In various embodiments of the present disclosure, the natural source may be a botanical or constituent or extract thereof, provided that if all compounds in the predetermined orthonasal profile are provided by a natural source, each natural source is not an oil derived from a botanical. For example, at least one of the chemical compound(s) in the predetermined orthonasal profile may be provided by an extract or constituent of a botanical, such as an extract or constituent from a Lamiaceae, Geraniaceae, Asteraceae, Myrtaceae or Ericaceae plant.

As used herein, the term “botanical” includes any material derived from a plant including, but not limited to, extracts, leaves, bark, fibres, stems, roots, seeds, flowers, fruits, pollen, husk, shells or the like. The material may be in the form of liquid, gas, solid, gel, powder, dust, crushed particles, granules, pellets, shreds, strips, sheets, or the like. In various embodiments of the present disclosure the material is in the form of liquid, solid, gel, powder, crushed particles, granules, pellets, shreds, strips, sheets or the like.

The Lamiaceae family are a family of flowering plants commonly known as the mint family. They may also be known as the Labiatae family. Many of the plants are aromatic in all parts and include widely used herbs such as basil, mentha, rosemary, sage, savory, marjoram, oregano, hyssop, thyme and lavender. At the time of writing, the last revision of the family was published in 2004 by Harley et al., “Labiatae” pages 167-275 of The Families and Genera of Vascular Plants volume VII. Springer-Verlag: Berlin; Heidelberg, Germany. This revision is incorporated herein by reference; it described and provided keys to 236 genera. A list at the Angiosperm Phylogeny website is also frequently updated: http://www.mobot.org/mobot/research/apweb/genera/lamiaceaegen.html. This list is incorporated herein by reference.

In various embodiments of the present disclosure, the Lamiaceae botanical is selected from members of the following genera: Acanthomintha, Achyrospermum, Acinos, Acrocephalus, Acrotome, Acrymia, Adelosa, Aegiphila, Aeollanthus, Agastache, Ajuga, Ajugoides, Alajja, Alvesia, Amasonia, Amethystea, Anisochilus, Anisomeles, Asterohyptis, Ballota, Basilicum, Becium, Benguellia, Blephilia, Bostrychanthera, Bovonia, Brachysola, Brazoria, Bystropogon, Calamintha, Callicarpa, Capitanopsis, Capitanya, Caryopteris, Catoferia, Cedronella, Ceratanthus, Chaiturus, Chamaesphacos, Chaunostoma, Chelonopsis, Chloanthes, Cleonia, Clerodendrum, Clinopodium, Colebrookea, Collinsonia, Colquhounia, Comanthosphace, Congea, Conradina, Coridothymus, Cornutia, Craniotome, Cryphia, Cuminia, Cunila, Cyanostegia, Cyclotrichium, Cymaria, Dauphinea, Dicerandra, Dicrastylis, Discretitheca, Dorystoechas, Dracocephalum, Drepanocaryum, Elsholtzia, Endostemon, Englerastrum, Eremostachys, Eriope, Eriophyton, Eriopidion, Eriothymus, Erythrochlamys, Euhesperida, Eurysolen, Faradaya, Fuerstia, Galeopsis, Garrettia, Geniosporum, Glechoma, Glechon, Glossocarya, Gmelina, Gomphostemma, Gontscharovia, Hanceola, Haplostachys, Haumaniastrum, Hedeoma, Hemiandra, Hemigenia, Hemiphora, Hemizygia, Hesperozygis, Heterolamium, Hoehnea, Holmskioldia, Holocheila, Holostylon, Horminum, Hosea, Hoslundia, Huxleya, Hymenocrater, Hymenopyramis, Hypenia, Hypogomphia, Hyptidendron, Hyptis, Hyssopus, Isodictyophorus, Isodon, Isoleucas, Kalaharia, Karomia, Keiskea, Killickia, Kudrjaschevia, Kurzamra, Lachnostachys, Lagochilus, Lagopsis, Lallemantia, Lamiophlomis, Lamium, Lavandula, Leocus, Leonotis, Leonurus, Lepechinia, Leucas, Leucophae, Leucosceptrum, Limniboza, Lophanthus, Loxocalyx, Lycopus, Macbridea, Madlabium, Marmoritis, Marrubium, Marsypianthes, Matsumurella, Meehania, Melissa, Melittis, Mentha, Meriandra, Mesona, Metastachydium, Microcorys, Micromeria, Microtoena, Minthostachys, Moluccella, Monarda, Monardella, Monochilus, Mosla, Neoeplingia, Neohyptis, Neorapinia, Nepeta, Newcastelia, Nosema, Notochaete, Obtegomeria, Ocimum, Octomeron, Ombrocharis, Oncinocalyx, Origanum, Orthosiphon, Otostegia, Ovieda, Oxera, Panzerina, Paralamium, Paraphlomis, Paravitex, Peltodon, Pentapleura, Perilla, Perillula, Peronema, Perovskia, Perrierastrum, Petitia, Petraeovitex, Phlomidoschema, Phlomis, Phlomoides, Phyllostegia, Physopsis, Physostegia, Piloblephis, Pitardia, Pityrodia, Platostoma, Plectranthus, Pogogyne, Pogostemon, Poliomintha, Prasium, Premna, Prostanthera, Prunella, Pseuderemostachys, Pseudocarpidium, Pseudocaryopteris, Pseudomarrubium, Puntia, Pycnanthemum, Pycnostachys, Rabdosiella, Renschia, Rhabdocaulon, Rhaphiodon, Rhododon, Rosmarinus, Rostrinucula, Rotheca, Roylea, Rubiteucris, Rydingia, Sabaudia, Saccocalyx, Salazaria, Salvia, Satureja, Schizonepeta, Schnabelia, Scutellaria, Sideritis, Siphocranion, Solenostemon, Spartothamnella, Sphenodesme, Stachydeoma, Stachyopsis, Stachys, Stenogyne, Sulaimania, Suzukia, Symphorema, Symphostemon, Synandra, Syncolostemon, Tectona, Teijsmanniodendron, Tetraclea, Tetradenia, Teucridium, Teucrium, Thorncroftia, Thuspeinanta, Thymbra, Thymus, Tinnea, Trichostema, Tripora, Tsoongia, Vitex, Viticipremna, Volkameria, Warnockia, Wenchengia, Westringia, Wiedemannia, Wrixonia, Xenopoma, Zataria, Zhumeria, and Ziziphora.

In various embodiments of the present disclosure, the natural source of at least one of the compounds in the predetermined orthonasal profile is a botanical or constituent or extract thereof, wherein the botanical is a member of the Lamiaceae family. In various embodiments of the present disclosure, the natural source of at least one of the compounds in the predetermined orthonasal profile is a botanical or constituent or extract thereof, wherein the botanical is a member of the Menthae tribe. The Menthae tribe is part of the Lamiaceae family and includes members of the following genera from the above list: Lycopus, Acanthomintha, Blephilia, Bystropogon, Clinopodium, Conradina, Cuminia, Cunila, Cyclotrichium, Dicerandra, Drymosiphon, Eriothymus, Glechon, Gontscharovia, Hedeoma, Hesperozygis, Hoehnea, Killickia (treated as part of Micromeria by Harley et al. 2004), Kurzamra, Mentha, Micromeria, Minthostachys, Monarda, Monardella, Obtegomeria, Origanum, Pentapleura, Piloblephis, Pogogyne, Poliomintha, Pycnanthemum, Rhabdocaulon, Rhododon, Saccocalyx, Satureja, Stachydeoma, Thymbra, Thymus, Zataria, Ziziphora, Agastache, Cedronella, Dracocephalum, Drepanocaryum, Glechoma, Heterolamium, Hymenocrater, Hyssopus, Kudrjaschevia, Lallemantia, Lophanthus, Marmoritis, Meehania, Nepeta, Schizonepeta, Cleonia, Horminum, Prunella, Chaunostoma, Dorystaechas, Lepechinia, Melissa, Meriandra, Neoeplingia, Perovskia, Rosmarinus, Salvia, Zhumeria, Acinos (treated as part of Clinopodium by Harley et al. 2004), Calamintha, and Pitardia.

In various embodiments of the present disclosure the natural source of at least one of the compounds in the predetermined orthonasal profile is a botanical or constituent or extract thereof, wherein the botanical is a member of the: Agastache, Mentha, Monarda, Minthostachys or Melissa genera, such as Agastache, Mentha, Monarda, Minthostachys or Melissa. In various embodiments of the present disclosure, the botanical is a member of the Mentha genus.

Agastache is a genus of aromatic flowing herbaceous perennial plants, which includes 22 species. As a whole, the genus is known as giant hyssops or hummingbird mints. The sections with their species are: Agastache sect. Agastache [Agastache cusickii, Agastache foeniculum, Agastache nepetoides, Agastache occidentalis, Agastache parvifolia, Agastache rugose, Agastache scrophulariifolia, Agastache urticifolia] and Agastache sect. Brittonastrum: [Agastache aurantiaca, Agastache breviflora, Agastache cana, Agastache coccinea, Agastache eplingiana, Agastache mearnsii, Agastache Mexicana, Agastache micrantha, Agastache pallida, Agastache pallidiflora, Agastache palmeri, Agastache pringlei, Agastache rupestris, Agastache wrightil]. In various embodiments of the present disclosure the Agastache botanical, or constituent or extract thereof, is a member of the Agastache rugose or Agastache rupestris species, e.g. Agastache rugose (Korean mint).

Throughout this disclosure the use of the term “species” and any specifically named species includes all subspecies, varieties, subvarieties, forms and subforms, along with any hybrids and/or cultivars thereof.

The Monarda genus includes the following species: Monarda bartletti, Monarda bradburiana, Monarda citriodora, Monarda clinopodia, Monarda clinopodioides, Monarda didyma, Monarda eplingiana, Monarda fistulosa, Monarda fruticulosa, Monarda humilis, Monarda lindheimeri, Monarda luteola, Monarda maritima, Monarda media, Monarda x medioides, Monarda pectinate, Monarda pringlei, Monarda punctate, Monarda russeliana, Monarda stanfieldii, Monarda viridissima. In various embodiments of the present disclosure the Monarda botanical, or constituent or extract thereof, is a member of the Monarda citriodora or Mondara didyma species, e.g. Monarda citriodora (lemon mint).

Melissa is a genus of perennial herbs and the most commonly grown species of this genus is Melissa officinalis, commonly known as lemon balm or balm. Other species include: Melissa axillaris Melissa flava and Melissa yunnanensis. In various embodiments of the present disclosure, the Melissa botanical or constituent or extract thereof is a member of the Melissa officinalis (lemon balm) species.

Mentha is a genus in which the distinction between species is more complex; it is estimated that 13 to 24 species exist because hybridization occurs naturally and some species overlap. As is known in the art, plant hybrids are generally given names that include an “x”, e.g. Mentha x piperita. Many cultivars also exist within the Mentha genus—an assemblage of plants selected for desirable characteristics that are maintained during propagation. Every unique cultivar has a unique name, which is regulated by the International Code of Nomenclature for Cultivated Plants; this name consists of a botanical name followed by a cultivar epithet, e.g. Mentha spicata ‘Russian’. As noted above, the present disclosure includes hybrids and cultivars. The botanical which is a member of the Mentha genus may therefore be a Mentha species, Mentha hybrid or Mentha cultivar.

In various embodiments of the present disclosure, at least one of the compounds in the orthonasal profile is provided by a botanical or constituent or extract thereof, wherein the botanical is a member of the Mentha genus and selected from the following species: Mentha alaica, Mentha aquatica, Mentha arvensis, Mentha atrolilacina, Mentha australis, Mentha balsamea, Mentha canadensis, Mentha cervina, Mentha cunninghamii, Mentha dahurica, Mentha darvasica, Mentha diemenica, Mentha gattefossei, Mentha grandiflora, Mentha haplocalyx, Mentha japonica, Mentha laxiflora, Mentha longifolia, Mentha micrantha, Mentha nemorosa, Mentha niliaca, Mentha pamiroalaica, Mentha pulegium, Mentha requienii, Mentha royleana, Mentha sachalinensis, Mentha satureioides, Mentha spicata, Mentha suaveolens, Mentha aquatica brevis, Mentha viridis, or a hybrid and/or cultivar thereof. The Mentha may be selected from the following species: Mentha alaica, Mentha aquatica, Mentha arvensis, Mentha atrolilacina, Mentha australis, Mentha canadensis, Mentha cervina, Mentha cunninghamii, Mentha dahurica, Mentha darvasica, Mentha diemenica, Mentha gattefossei, Mentha grandiflora, Mentha japonica, Mentha laxiflora, Mentha longifolia, Mentha micrantha, Mentha nemorosa, Mentha niliaca, Mentha pamiroalaica, Mentha pulegium, Mentha requienii, Mentha royleana, Mentha sachalinensis, Mentha satureioides, Mentha suaveolens, Mentha aquatica brevis, or a hybrid and/or cultivar thereof.

Recognized hybrids of the Mentha genus include: Mentha x carinthiaca (M. arvensis x M. suaveolens), Mentha x dalmatica (M. arvensis x M. longifolia), Mentha x dumetorum (M. aquatica x M. longifolia), Mentha x gayeri, Mentha x gentilis, (syn. Mentha x gracilis; M. arvensis x M. spicata), Mentha x kuemmerlei, Mentha x locyana, Mentha x piperita (M. aquatica x M. spicata), Mentha x pyramidalis, Mentha x rotundifolia (M. longifolia x M. suaveolens), Mentha x suavis, (syn. Mentha x maximilianea; M. aquatica x M. suaveolens), Mentha x verticillata (M. aquatica x M. arvensis), Mentha x villosa (M. spicata x M. suaveolens, also called M. nemorosa), Mentha x villosa-nervata, (M. longifolia x M. spicata), Mentha x wirtgeniana (syn. Mentha x smithiana; M. aquatica x M. arvensis x M. spicata), and cultivars thereof.

Cultivars within the Mentha genus are numerous and include e.g. Mentha suaveolens Variegata, Mentha x piperita f. citrata ‘Lemon’, Mentha x piperita f. citrata ‘Basil’, Mentha x piperita f. citrata ‘Lime’, Mentha x piperita f. citrata ‘Grapefruit’, Mentha spicata subsp. citrata ‘Strawberry’, Mentha x piperita f. citrata ‘Orange’, Mentha x piperita f. citrata ‘Chocolate’, Mentha x piperita‘Lavender’, Mentha x gracilis ‘Variegata’, Mentha arvensis‘Banana’, Mentha spicata var. crispa ‘Moroccan’, Mentha spicata var. crispa ‘Persian’, Mentha spicata ‘Russian’, Mentha piperita ‘Canyon’, Mentha ‘wild berries’, Mentha ‘sweet pear’, Mentha ‘Iced hazelnut’, Mentha ‘Berries and Cream’, Mentha ‘Pink Candypops’, Mentha ‘Marshmallow’, Mentha ‘Oregano-Thyme’, Mentha ‘Sister Julie's Wintergreen’, Mentha ‘Jim's Candy Lime’, Mentha ‘Margarita’, and Mentha ‘Sweet Bay’.

The present disclosure is not, however, limited to these species, hybrids or cultivars or in fact the Mentha genus. In various embodiments of the present disclosure, at least one of the compounds in the predetermined orthonasal profile is provided by a botanical, or constituent or extract thereof, wherein the botanical is selected from members of the Geraniaceae, Asteraceae, Myrtaceae, Ericaceae or Lamiaceae families but is not a member of the Mentha genus.

The members of the Lamiaceae family other than members of the Mentha genus are discussed above. This discussion is generally applicable, and includes for example, Agastache (e.g. Agastache rugose, Agastache rupestris ‘Apache Sunset’), Calamintha (e.g. Calamintha Nepeta), Pycnanthemum (e.g. Pycnanthemum pilosum), Micromeria (e.g. Micromeria fruticose), Salvia (e.g. Salvia elegans, Salvia elegans ‘Tangerine’, Salvia dorisiana, Salvia elegans‘Honey Melon’, Salvia discolor, Salvia melissodora), Ocimum (e.g. Ocimum basilicum ‘cinnamon’, Ocimum basilicum ‘anise’ Ocimum sellol) or Thymus (e.g. Thymus sp., Thymus ‘TM122’, Thymus sp).

In various embodiments of the present disclosure, at least one of the compounds in the predetermined orthonasal profile is provided by a botanical, or constituent or extract thereof, wherein the botanical is selected from members of the Geraniaceae family. Geraniaceae is a family of flowering plants placed in the order Geraniales. The family comprises 830 species in five to seven genera, the largest genera are Geranium, Pelargonium and Erodium. In various embodiments, the Geraniaceae botanical or constituent or extract thereof is selected from members of the Pelargonium genus. The Pelargonium genus has between 200 and 280 species. At the time of writing, the Plant List accepted 250 species at http://www.theplantlist.org/1.1/browse/A/Geraniaceae/Pelargonium/—this list is incorporated herein by reference.

In various embodiments of the present disclosure the Geraniaceae botanical or constituent or extract thereof is selected from Pelargonium species, hybrids or cultivars. Cultivated pelargoniums are commonly divided into six groups: A) Zonal, B) Ivy-leaved, C) Regal, D) Angel, E) Unique, F) Scented-leaved, G) Species, and H) Primary hybrids. In various embodiments of the present disclosure the Pelargonium botanical or constituent or extract thereof is selected from members of group F. Group F are perennials grown chiefly for their scent and include:
Almond—Pelargonium quercifolium; Apple—Pelargonium odoratissimum;
Apple—Pelargonium cordifolium; Apple/Mint—Pelargonium album;
Apricot/Lemon—Pelargonium scabrum; Balsam—Pelargonium panduriforme;
Camphor—Pelargonium botulinum; Celery—Pelargonium ionidiflorum;
Cinnamon—Pelargonium ‘Ardwyck Cinnamon’; Coconut—Pelargonium grossalarioides (Pelargonium parriflorum); Eau de Cologne—Pelargonium ‘Brilliantine’;
Eucalyptus—Pelargonium ‘Secret Love’; Grapefruit—Pelargonium ‘Poquita’;
Ginger—Pelargonium ‘Torrento’ or ‘Cola Bottles’ which is a variety of Pelargonium x nervosum;
Hazelnut—Pelargonium ‘Odorata Hazelnut; Lavender—Pelargonium ‘Lavender Lindy’; Lemon
Pelargonium crispum; Lemon—Pelargonium citronellum (Synonym—Pelargonium ‘Mabel Grey’); Lemon Balm—Pelargonium x melissinum;
Lime—Pelargonium x nervosum; Myrrh—Pelargonium myrrhifolium; Nutmeg—Pelargonium x fragrans; Old Spice—Variety of Pelargonium x fragrans; Orange—Pelargonium x citriodorum (Synonym—Pelargonium ‘Prince of Orange’); Peach—Pelargonium ‘Peaches and Cream’;
Peppermint—Pelargonium tomentosum; Pine—Pelargonium denticulatum;
Pineapple—Pelargonium ‘Brilliant’; Raspberry—Pelargonium ‘Red Raspberry’;
Rose—Pelargonium graveolens (Synonym—Pelargonium roseum); Rose—Pelargonium capitatum; Rose—Pelargonium radens; Southernwood—Pelargonium abrotanifolium;
Spicy—Pelargonium exstipulatum; Strawberry—Pelargonium x scarboroviae; and the cultivars: ‘Attar of Roses’—a cultivar of P. capitatum; ‘Crowfoot Rose’—a cultivar of P. radens; ‘Dr. Livingston’—a cultivar of P. radens; ‘Grey Lady Plymouth’—a cultivar of P. graveolens; and ‘Prince Rupert’—a cultivar of P. crispum.

In various embodiments of the present disclosure, the Pelargonium botanical or constituent or extract thereof is selected from Perlargonium ‘Pungent Peppermint’, and Pelargonium x scarboroviae.

In various embodiments of the present disclosure, at least one of the compounds in the predetermined orthonasal profile is provided by a botanical, or constituent or extract thereof, wherein the botanical is selected from members of the Asteraceae family. Asteraceae (otherwise known as Compositae) is a large family of flowering plants. The family includes over 32,000 currently accepted species in over 1,900 genera in 13 subfamilies. In various embodiments of the present disclosure the Asterceae botanical, or constituent or extract thereof, is selected from members of the Asteroideae subfamily. The Asteroideae subfamily is made up of several tribes including Astereae, Calenduleae, Eupatorieae, Gnaphalieae, Heliantheae, Senecioneae and Tageteae; these tribes are grouped into three supertribes: Senecionodae, Asterodae and Helianthodae. In various embodiments of the present disclosure, the Asterceae botanical, or constituent or extract thereof, is selected from members of the Helianthodae supertribe of the Asteroideae subfamily, e.g. the Tageteae tribe. The Tageteae tribe consists of approximately 216 species divided among 28 genera including: Adenophyllum, Bajacalia, Boeberastrum, Boeberoides, Chrysactinia, Clappia, Comaclinium, Coulterella, Dysodiopsis, Dyssodia, Gymnolaena, Haploesthes, Harnackia, Hydropectis, Lescaillea, Leucactinia, Jamesianthus, Jaumea, Nicolletia, Oxypappus, Pectis, Porophyllum, Pseudoclappia, Sartwellia, Schizotrichia, Strotheria, Tagetes, Thymophylla, Urbinella, and Varilla. In various embodiments of the present disclosure the Asterceae botanical, or constituent or extract thereof is selected from members of the Tagetes genera of the Tageteae family, for example, Tagetes lemmonii.

In various embodiments of the present disclosure, at least one of the compounds in the predetermined orthonasal profile is provided by a botanical, or constituent or extract thereof, wherein the botanical is selected from members of the Myrtaceae family. Myrtaceae is a family of dicotyledonous plants placed within the order Myrtales. Myrtle, pohutukawa, bay rum tree, clove, guava, acca (feijoa), allspice, and eucalyptus are some notable members of this group.

All species are woody and contain essential oils. In various embodiments of the present disclosure the Myrtaceae botanical, or constituent or extract thereof, is selected from members of the subfamily Myrtoideae, e.g. Myrtae or Eucalypteae.

In various embodiments of the present disclosure at least one of the compounds in the predetermined orthonasal profile is provided by a botanical, or constituent or extract thereof, wherein the botanical is selected from members of Eucalypteae, for example members of a genera selected from: Allosyncarpia, Angophora, Arillastrum, Corymbia, Eucalyptopsis, Eucalyptus, and Stockwellia. In various embodiments of the present disclosure, at least one of the compounds in the predetermined orthonasal profile is provided by a botanical, or constituent or extract thereof, wherein the botanical is selected from members of the Eucalyptus genus, for example, Eucalyptus radiata.

In various embodiments of the present disclosure, at least one of the compounds in the predetermined orthonasal profile is provided by a botanical, or constituent or extract thereof, wherein the botanical is selected from members of the Ericaceae family. The Ericaceae family includes 9 subfamilies: Enkianthoideae (one genus, 16 species), Pyroloideae (4 genera, 40 species), Monotropoideae (10 genera, 15 species), Arbutoideae (up to six genera, about 80 species), Cassiopoideae (one genus, 12 species), Ericoideae (19 genera, 1790 species), Harrimanelloideae (one genus, two species), Styphelioideae (35 genera, 545 species), and Vaccinioideae (50 genera, 1580 species). In various embodiments of the present disclosure at least one of the compounds in the predetermined orthonasal profile is provided by a botanical, or constituent or extract thereof, wherein the botanical is selected from members of the Vaccinioideae subfamily of Ericaceae; this family contains e.g. cranberry, blueberry, bilberry, lingonberry and huckleberry.

One of the genera of the Vaccinioideae subfamily is Gaultheria; this includes about 135 species including American wintergreen or Gaultheria procumbens. In various embodiments of the present disclosure at least one of the compounds in the predetermined orthonasal profile is provided by a botanical, or constituent or extract thereof, wherein the botanical is selected from members of the genus Gaultheria, e.g. Gaultheria procumbens.

In various embodiments of the present disclosure, at least one of the compounds in the predetermined orthonasal profile is provided by a botanical, or constituent or extract thereof, wherein the botanical is independently selected from the group consisting of: Mentha x piperita, Mentha viridis, Mentha spicata, Mentha balsamea, Mentha haplocalyx, Mentha suaveolens, Mentha suaveolens Variegata, Melissa officinalis, Mentha x piperita f. citrata ‘Lemon’, Mentha x piperita f. citrata ‘Basil’, Mentha x piperita f. citrata ‘Lime’, Mentha x piperita f. citrata ‘Grapefruit’, Mentha spicata subsp. citrata ‘Strawberry’, Mentha x piperita f. citrata ‘Orange’, Mentha x piperita f. citrata, Monarda didyma, Mentha x piperita f. citrata ‘Chocolate’, Mentha x piperita ‘Lavender’, Mentha x gracilis, Mentha x gracilis ‘Variegata’, Mentha arvensis ‘Banana’, Mentha aquatica, Mentha x villosa, Mentha nemorosa, Mentha spicata var. crispa ‘Moroccan’, Mentha sachalinensis, Agastache rugose, Mentha spicata var. crispa‘Persian’, Mentha spicata ‘Russian’, Mentha requienii, Mentha arvensis, Mentha arvensis L. var. canandensis, Mentha canadensis, Mentha niliaca, Calamintha Nepeta, Mentha australis, Mentha piperita ‘Canyon’, Mentha ‘wild berries’, Mentha ‘sweet pear’, Mentha laxiflora, Mentha ‘Iced hazelnut’, Mentha ‘Berries and Cream’, Mentha ‘Pink Candypops’, Mentha ‘Marshmallow’, Mentha ‘Oregano-Thyme’, Mentha ‘Sister Julie's Wintergreen’, Mentha ‘Jim's Candy Lime’, Mentha ‘Margarita’, Mentha ‘Sweet Bay’, Pycnanthemum pilosum, Micromeria fruticose, Perlargonium ‘Pungent Peppermint’, Pelargonium x scarboroviae, Agastache rupestris ‘Apache Sunset’, Salvia elegans, Salvia elegans ‘Tangerine’, Eucalyptus radiata, Tagetes lemmonii, Salvia dorisiana, Salvia elegans ‘Honey Melon’, Salvia discolor, Salvia melissodora, Ocimum basilicum ‘cinnamon’, Ocimum basilicum ‘anise’, Ocimum selloi, Thymus sp., Thymus ‘TM122’, Thymus sp., Gaultheria procumbens, or hybrids or cultivars thereof.

In various embodiments of the present disclosure, at least one of the compounds in the predetermined orthonasal profile is provided by a botanical, or constituent or extract thereof, wherein the botanical is independently selected from the group consisting of: Mentha xpiperita, Mentha viridis, Mentha spicata, Mentha balsamea, Mentha haplocalyx, Mentha suaveolens, Melissa officinalis, Mentha x piperita f. citrata, Mentha spicata subsp. citrata, Monarda didyma, Mentha x gracilis, Mentha x gracilis, Mentha arvensis, Mentha aquatica, Mentha x villosa, Mentha nemorosa, Mentha spicata var. crispa, Mentha sachalinensis, Agastache rugose, Mentha Persian, Mentha requienii, Mentha arvensis L. var. canadensis, Mentha canadensis, Mentha niliaca, Calamintha Nepeta, Mentha australis, Mentha laxiflora, hybrids and/or cultivars thereof, or a combination thereof.

In various embodiments of the present disclosure, at least two of the compounds in the predetermined orthonasal profile are each provided by a botanical, or constituent or extract thereof, wherein each botanical is independently selected from the group consisting of: Mentha x piperita, Mentha viridis, Mentha spicata, Mentha balsamea, Mentha haplocalyx, Mentha suaveolens, Mentha suaveolens Variegata, Melissa officinalis, Mentha x piperita f. citrata ‘Lemon’, Mentha x piperita f. citrata ‘Basil’, Mentha x piperita f. citrata ‘Lime’, Mentha x piperita f. citrata ‘Grapefruit’, Mentha spicata subsp. citrata ‘Strawberry’, Mentha x piperita f. citrata ‘Orange’, Mentha x piperita f. citrata, Monarda didyma, Mentha x piperita f. citrata ‘Chocolate’, Mentha x piperita ‘Lavender’, Mentha x gracilis, Mentha x gracilis ‘Variegata’, Mentha arvensis ‘Banana’, Mentha aquatica, Mentha x villosa, Mentha nemorosa, Mentha spicata var. crispa ‘Moroccan’, Mentha sachalinensis, Agastache rugose, Mentha spicata var. crispa ‘Persian’, Mentha spicata ‘Russian’, Mentha requienii, Mentha arvensis, Mentha arvensis L. var. canadensis, Mentha canadensis, Mentha niliaca, Calamintha Nepeta, Mentha australis, Mentha piperita ‘Canyon’, Mentha ‘wild berries’, Mentha ‘sweet pear’, Mentha laxiflora, Mentha ‘Iced hazelnut’, Mentha ‘Berries and Cream’, Mentha ‘Pink Candypops’, Mentha ‘Marshmallow’, Mentha ‘Oregano-Thyme’, Mentha ‘Sister Julie's Wintergreen’, Mentha ‘Jim's Candy Lime’, Mentha ‘Margarita’, Mentha ‘Sweet Bay’, Pycnanthemum pilosum, Micromeria fruticose, Perlargonium ‘Pungent Peppermint’, Pelargonium x scarboroviae, Agastache rupestris ‘Apache Sunset’, Salvia elegans, Salvia elegans ‘Tangerine’, Eucalyptus radiata, Tagetes lemmonii, Salvia dorisiana, Salvia elegans ‘Honey Melon’, Salvia discolor, Salvia melissodora, Ocimum basilicum ‘cinnamon’, Ocimum basilicum ‘anise’, Ocimum selloi, Thymus sp., Thymus ‘TM122’, Thymus sp., Gaultheria procumbens, or hybrids or cultivars thereof.

In various embodiments of the present disclosure, at least two of the compounds in the predetermined orthonasal profile are each provided by a botanical, or constituent or extract thereof, wherein each botanical is independently selected from the group consisting of: Mentha x piperita, Mentha viridis, Mentha spicata, Mentha balsamea, Mentha haplocalyx, Mentha suaveolens, Melissa officinalis, Mentha x piperita f. citrata, Mentha spicata subsp. citrata, Monarda didyma, Mentha x gracilis, Mentha x gracilis, Mentha arvensis, Mentha aquatica, Mentha x villosa, Mentha nemorosa, Mentha spicata var. crispa, Mentha sachalinensis, Agastache rugose, Mentha requienii, Mentha arvensis L. var. canadensis, Mentha canadensis, Mentha niliaca, Calamintha Nepeta, Mentha australis, Mentha laxiflora, hybrids and/or cultivars thereof, or a mixture thereof.

The cultivars of the above-mentioned species are not limited and can, for example include: Mentha spicata var. crispa Persian’, Mentha suaveolens ‘Variegata’, Mentha x piperita f. citrata ‘Lemon’, Mentha x piperita f. citrata ‘Basil’, Mentha x piperita f. citrata ‘Lime’, Mentha x piperita f. citrata ‘Grapefruit’, Mentha spicata subsp. citrata ‘Strawberry’, Mentha x piperita f. citrata ‘Orange’, Mentha x piperita f. citrata ‘Chocolate’, Mentha x piperita ‘Lavender’, Mentha x gracilis ‘Variegata’, Mentha arvensis‘Banana’, Mentha spicata var. crispa‘Moroccan’, Mentha spicata ‘Russian’, Mentha piperita ‘Canyon’, Mentha ‘wild berries’, Mentha ‘sweet pear’, Mentha ‘Iced hazelnut’, Mentha ‘Berries and Cream’, Mentha ‘Pink Candypops’, Mentha ‘Marshmallow’, Mentha ‘Oregano-Thyme’, Mentha ‘Sister Julie's Wintergreen’, Mentha ‘Jim's Candy Lime’, Mentha ‘Margarita’, Mentha ‘Sweet Bay’ or a combination thereof.

In various embodiments of the present disclosure, at least two of the predetermined orthonasal profile are each provided by a botanical, or constituent, or extract thereof, wherein each botanical is independently selected from the group consisting of: Mentha x piperita, Mentha viridis, Mentha spicata, Mentha balsamea, Mentha haplocalyx, Mentha suaveolens, Mentha suaveolens Variegata, Melissa officinalis, Mentha x piperita f. citrata‘Lemon’, Mentha x piperita f. citrata ‘Basil’, Mentha x piperita f. citrata ‘Lime’, Mentha x piperita f. citrata ‘Grapefruit’, Mentha spicata subsp. citrata ‘Strawberry’, Mentha x piperita f. citrata ‘Orange’, Mentha x piperita f. citrata, Monarda didyma, Mentha x piperita f. citrata ‘Chocolate’, Mentha x piperita ‘Lavender’, Mentha x gracilis, Mentha x gracilis ‘Variegata’, Mentha arvensis ‘Banana’, Mentha aquatica, Mentha x villosa, Mentha nemorosa, Mentha spicata var. crispa ‘Moroccan’, Mentha sachalinensis, Agastache rugose, Mentha Persian, Mentha spicata ‘Russian’, Mentha requienii, Mentha arvensis, Mentha arvensis L. var. canandensis, Mentha canadensis, Mentha niliaca, Calamintha Nepeta, Mentha australis, Mentha piperita‘Canyon’, Mentha ‘wild berries’, Mentha ‘sweet pear’, Mentha laxiflora, Mentha ‘Iced hazelnut’, Mentha ‘Berries and Cream’, Mentha ‘Pink Candypops’, Mentha ‘Marshmallow’, Mentha ‘Oregano-Thyme’, Mentha ‘Sister Julie's Wintergreen’, Mentha ‘Jim's Candy Lime’, Mentha ‘Margarita’, Mentha ‘Sweet Bay’, or a combination thereof.

In various embodiments of the present disclosure, at least two of the compounds in the predetermined orthonasal profile are each provided by a botanical, or constituent or extract thereof, wherein each botanical is independently selected from the group consisting of: Mentha x piperita, Mentha viridis, Mentha spicata, Mentha balsamea, Mentha haplocalyx, Mentha suaveolens, Mentha suaveolens Variegata, Melissa officinalis, Mentha x piperita f. citrata ‘Lemon’, Mentha x piperita f. citrata ‘Basil’, Mentha x piperita f. citrata ‘Lime’, Mentha x piperita f. citrata ‘Grapefruit’, Mentha spicata subsp. citrata ‘Strawberry’, Mentha x piperita f. citrata ‘Orange’, Mentha x piperita f. citrata, Monarda didyma, Mentha x piperita f. citrata ‘Chocolate’, Mentha x piperita ‘Lavender’, Mentha x gracilis, Mentha x gracilis ‘Variegata’, Mentha arvensis ‘Banana’, Mentha aquatica, Mentha x villosa, Mentha nemorosa, Mentha spicata var. crispa‘Moroccan’, Mentha sachalinensis, Agastache rugose, Mentha Persian, Mentha spicata ‘Russian’, Mentha requienii, Mentha arvensis, Mentha arvensis L. var. canandensis, Mentha canadensis, Mentha niliaca, Calamintha Nepeta, Mentha australis, Mentha piperita ‘Canyon’, Mentha ‘wild berries’, Mentha ‘sweet pear’, Mentha laxiflora, Mentha ‘Iced hazelnut’, Mentha ‘Berries and Cream’, Mentha ‘Pink Candypops’, Mentha ‘Marshmallow’, Mentha ‘Oregano-Thyme’, Mentha ‘Sister Julie's Wintergreen’, Mentha ‘Jim's Candy Lime’, Mentha ‘Margarita’, Mentha ‘Sweet Bay’, or a combination thereof.

For example, at least one of the botanicals, or constituent or extract thereof, may be selected from one or more (e.g. one) of the following groups:
Mentha aquatica, Mentha x piperita f. citrata ‘Lemon’,
Mentha x piperita ‘Lavender’, Mentha spicata, Mentha suaveolens ‘Variegata’,
Mentha x piperita f. citrata ‘Grapefruit’, Mentha x gracilis ‘Variegata’,
Mentha x piperita, Mentha x villosa,
or a combination thereof.
Mentha x piperita, Mentha suaveolens, Mentha x piperita f. citrata ‘Orange’, Mentha x
piperita f. citrata ‘Lime’, Mentha spicata,
Mentha spicata subsp. citrata ‘Strawberry’,
or a combination thereof.
Mentha australis, Mentha x piperita f. citrata ‘Orange’, Mentha x piperita ‘Canyon’,
Mentha suaveolens ‘Variegata’, Mentha x piperita f. citrata ‘Basil’,
Mentha spicata, Mentha x piperita f. citrata ‘Chocolate’, Mentha ‘wild berries’,
or a combination thereof.
Mentha spicata var. crispa ‘Moroccan’, Mentha suaveolens, Mentha Persian, Mentha x piperita f. citrata ‘Lemon’, Mentha x gracilis ‘Variegata’,
Mentha x villosa, Mentha x piperita f. citrata ‘Lime’,
Mentha suaveolens ‘Variegata’,
or a combination thereof.
Mentha x piperita, Mentha x piperita f. citrata ‘Chocolate’, Mentha ‘sweet pear’,
Mentha spicata, Mentha arvensis ‘Banana’, Mentha x piperita citrata,
Mentha ‘wild berries’, Mentha x piperita f. citrata ‘Lemon’,
Mentha x piperita f. citrata ‘Chocolate’,
or a combination thereof.
Mentha x piperita, Mentha x piperita f. citrata ‘Grapefruit’,
Mentha x piperita f. citrata ‘Lime’, Mentha spicata,
Mentha x piperita f. citrata ‘Lemon’, Mentha spicata subsp. citrata ‘Strawberry’,
Mentha suaveolens ‘Variegata’, Mentha x piperita citrata, Mentha ‘sweet pear’,
Mentha x piperita ‘Lavender’,
or a combination thereof.

Accordingly the composition obtainable from the method of the present disclosure may have an aroma in addition to mint which is provided by a first botanical or constituent, or extract thereof and/or a second botanical or constituent, or extract thereof. This concept is supported by the various species, hybrids and cultivars discussed herein, and improves the flexibility of the manufacturer when looking to prepare a mint-based product for use in a substance delivery system such as an e-cigarette, tobacco-heating device, smoking article or oral product.

It will be understood by the person skilled in the art that the present disclosure is not limited to two botanicals but includes an unlimited number or further botanicals or constituents, or extracts thereof to build the desired orthonasal profile for the flavoured composition. The further botanicals that may be included are not necessarily limited although they are in various embodiments, selected from members of the Lamiaceae, Geraniaceae, Asteraceae, Myrtaceae or Ericaceae families. The further botanicals, including the third botanical, may be defined in the same manner as the botanicals discussed hereinabove.

By the expression “constituent or extract” is meant a component isolated from the botanical. In various embodiments of the present disclosure, the component isolated from the botanical is a natural extract, for example an oil (often referred to as an essential oil) or its constituents. Such extracts may be available commercially or may be obtained by methods known in the art. The person skilled in the art will be aware of suitable methods in this respect. As noted above, if all the chemical compounds in the predetermined orthonasal profile are from a natural source, they are not all from oils extracted from a botanical. A compound extracted from a botanical may be included in any application of the consumable. In various embodiments such a compound may be included in the consumable when used in an oral product described herein as well as a tobacco heating device or an aerosol generating system.

In various embodiments of the present disclosure at least one of the chemical compound(s) is provided by a synthetic source. The synthetic source is not limited and may include any source known in the art. including commercially available and synthetically obtained compounds. Compounds may, for instance, be obtained from Sigma Aldrich (https://www.sigmaaldrich.com/united-kingdom.html) or the like.

In various embodiments of the present disclosure, one or more compound(s) in the predetermined orthonasal profile may be an active compound naturally existing in a botanical which is obtained synthetically. The compound may be commercially available or obtained by synthetic methods known in the art; again, the person skilled in the art will be aware of suitable methods in this respect.

### Flavoured Composition

The present disclosure provides a composition obtainable from the method of the second aspect. Said composition has a primary flavour, at least a first aroma associated with the primary flavour and at least an additional or second aroma as discussed above. The composition is novel and inventive in its own right because of the unique orthonasal profile delivered to a consumer.

In the example embodiment, a mint-flavoured composition is provided which has at least one aroma other than mint. The mint-flavoured composition may further comprise one or more sensate compound, the sensate compound(s) being defined hereinbelow. The present disclosure also relates to consumable including inter alia the flavoured (e.g. mint-flavoured) composition.

By the term “mint-flavoured” is meant the user of the consumable in a substance delivery system experiences a mint (or minty) taste, smell and feeling from the composition. This is the primary flavour of the composition. The term “flavour” is defined above. A mint flavour is typically described as “fresh”, “cool”, “cold”, “refreshing” and/or “clean”; it can be described as a combination of a sweet taste, a fresh, aromatic aroma and a cooling aftertaste or sensation. A “mint-flavour” or “minty flavour” would be readily identified by the person skilled in the art.

The expression “aroma other than the primary flavour” or “second aroma which is different from the first aroma” is discussed above. All expressions are intended to mean a scent, smell, aroma or aromatic other than and different from that associated with the primary flavour.

Unlike the currently available flavours for substance delivery systems such as e-cigarettes, tobacco heating devices, smoking articles or oral products, the composition of the present disclosure provides a unique sensation by delivering a multi-sensorial experience. Not only is the user provided with the desired flavour, but they receive a further sensation from the additional aroma(s). The additional aroma(s) can be chosen to stimulate a desired sensation in a user, for example: a refreshing taste of grapefruit, warm notes of ginger, floral lavender notes, hints of lemon and lime zest, sweet citrus etc. It allows the manufacturer to enhance the user experience and broaden the products available to consumers.

The identity of the additional aroma(s) is not limited and can include any scent known in the art. Exemplary additional aromas include: apple, pineapple, chocolate, berry (e.g. raspberry, blueberry, strawberry), grapefruit, ginger, banana, orange, lemon, lime, pear, mango, basil, cinnamon, coffee, lavender, oregano, wintergreen, coconut, melon, blackcurrant, grape, anise, rose, licorice, root beer, coconut, grape, green pepper, hazelnut, marshmallow, peach, thyme, basil, oregano, bay leaf and bergamot. As the person skilled in the art will appreciate, the additional aroma may be a combination of scents, with one scent being more dominant than another. The present disclosure is not limited in this respect; all that is required is for the additional aroma to be identifiable alongside the primary flavour.

In various embodiments of the present disclosure, the additional aroma or aroma other than the primary flavour (e.g. mint) is selected from the group consisting of: citrus, fruity, floral, green, herbal, vegetable, creamy, roasted, nutty, woody, spicy, cheesy or a combination thereof. In various embodiments of the present disclosure, the additional aroma is selected from citrus, fruity, herbal, floral, creamy (e.g. chocolate), roasted, nutty or a combination thereof.

In various embodiments of the present disclosure, the additional aroma is selected from the group consisting of: lemon, bergamot, orange, floral, grapefruit, lime, banana, pineapple, apple, pear, mango, basil, cinnamon, tropical, chocolate, honey, grass, clove, vanilla, strawberry, raspberry, ginger, lavender, coconut, melon, blackcurrant, grape, anise, rose, liquorice, root beer, coconut, grape, green pepper, hazelnut, marshmallow, peach, thyme, basil, oregano, bay leaf, or a combination thereof.

In various embodiments of the present disclosure the additional aroma is selected from the group consisting of: apple, floral, pineapple, chocolate, berry (e.g. raspberry, blueberry, strawberry), grapefruit, ginger, banana, orange, lemon, lime, pear, mango, basil, cinnamon, coffee, lavender, oregano, wintergreen and bergamot, or a mixture thereof. For example the additional aroma may be selected from the group consisting of: lemon, bergamot, orange, floral, grapefruit, lime, banana, pineapple, apple, pear, mango, basil, cinnamon, chocolate, strawberry, raspberry, ginger, lavender, or a mixture thereof.

The form of the flavoured composition is not limited and will typically depend on the substance delivery system in which it is intended to be used. The composition may be a liquid, solid or gel. The composition may, for example, be a powder, dust, crushed particles, granules, pellets, shreds, strips, sheets, or the like. In various embodiments of the present disclosure, the composition is in the form of a liquid, solid or gel. In various embodiments of the present disclosure, the composition is in the form of a liquid or gel.

The flavoured composition may also be loaded onto a substrate material within the consumable. The substrate or support may, for example, be or comprise a carrier material such as paper, card, paperboard, cardboard, tobacco, reconstituted material (e.g. reconstituted tobacco), a plastics material, a ceramic material, a composite material, a polymeric (e.g. fibrous) material, glass, a metal, or a metal alloy. Substrate materials are discussed in more detail below.

### Consumable

The consumable according to the present disclosure is for use in a substance delivery system and includes at least one further component selected from aerosol-former materials, active substances, substrate materials, or a combination thereof. The consumable or the flavoured composition may further comprise one or more additional flavours, one or more sensate compounds and/or one or more other functional materials. Often these components will depend on the substance delivery system in which the consumable is intended to be used.

The consumable is an article comprising, consisting essentially of or consisting of the flavoured composition as defined herein and the further components listed in the claims, part of all of which is intended to be consumed during use of the substance delivery system by a user. The term “substance delivery system” is intended to encompass systems that deliver at least one substance to a user. In some embodiments, the substance to be delivered may be the composition itself. In other embodiments the substance to be delivered may be a different material to the composition: either an aerosol-generating material or a material that is not intended to be aerosolised. As appropriate, either material may comprise the one or more active constituents, one or more aerosol-former materials, one or more substrate materials.

In some embodiments, the flavoured composition is present in an amount of from about 0.001% to about 95% by weight of the consumable. In some embodiments, the flavoured composition is present in an amount of from about 0.1% to about 80% by weight of the consumable, such as 1% to about 70% by weight or 10% by weight to about 60% by weight.

In other embodiments, the flavoured composition is present in an amount from about 0.001% to about 20% by weight of the consumable. In some embodiments, the flavoured composition is present in an amount of from about 0.01% to about 15% by weight of the consumable, such as from about 0.1% to about 10% by weight of the consumable, such as from about 0.5% to about 7.5% by weight of the consumable, such as from about 1% to about 5% by weight of the consumable. In some embodiments, the flavoured composition is present in an amount of from about 0.1% to about 5% by weight of the consumable, such as from about 0.25% to about 4.5% by weight of the consumable, such as from about 5% to about 4% by weight of the consumable, such as from about 0.75% to about 3.5% by weight of the consumable, such as from about 1% to about 3% by weight of the consumable, such as from about 1.5% to about 2.5% by weight of the consumable. In some embodiments, the flavoured composition is present in an amount of from about 0.001% to about 2% by weight of the consumable, such as from about 0.01% to about 1.5% by weight of the consumable, such as from about 0.05% to about 1% by weight of the consumable, such as from about 0.1% to about 0.75% by weight of the consumable, such as from about 0.1% to about 0.5% by weight of the consumable. The skilled person will appreciate that these amounts can be combined with the amounts of the other components making up the consumable, e.g. the aerosol-former material, active substance and/or the substrate material.

The active substance as used herein may be a physiologically active material, which is a material intended to achieve or enhance a physiological response. The active substance may be selected from nutraceuticals, nootropics, and psychoactives. The active substance may be naturally occurring or synthetically obtained. The active substance may comprise for example nicotine, caffeine, taurine, theine, vitamins such as B6 or B12 or C, melatonin, cannabinoids, or constituents, derivatives, or combinations thereof. The active substance may comprise one or more constituents, derivatives or extracts of tobacco, cannabis or another botanical.

In some embodiments, the active substance comprises nicotine or a salt thereof (e.g. nicotine ditartrate/nicotine bitartrate). In some embodiments, the active substance comprises caffeine, melatonin or vitamin B12. In various embodiments of the present disclosure, the flavoured composition or the consumable comprises an active substance which is nicotine or a salt thereof. In other embodiments, the flavoured composition or the consumable is nicotine-free. A nicotine-free composition may include nicotine-free tobacco substitutes.

As noted herein, the active substance may comprise one or more constituents, derivatives or extracts of cannabis, such as one or more cannabinoids or terpenes. Cannabinoids are a class of natural or synthetic chemical compounds which act on cannabinoid receptors (i.e., CB1 and CB2) in cells that repress neurotransmitter release in the brain. Cannabinoids are cyclic molecules exhibiting particular properties such as the ability to easily cross the blood-brain barrier. Cannabinoids may be naturally occurring (Phytocannabinoids) from plants such as cannabis, (endocannabinoids) from animals, or artificially manufactured (synthetic cannabinoids). Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

In various embodiments of the present disclosure, the active substance is a cannabinoid such as cannabidiol (CBD), tetrahydrocannabinol (THC), or pharmaceutically acceptable salts thereof. In various embodiments, the cannabidiol or tetrahydrocannabinol is synthetic.

The active substance may be present in any suitable amount depending on the desired dosage when consumed by the user. In various embodiments, the active substance may be present in an amount of about 0.01% by weight to about 10% by weight of the consumable. In some embodiments, the active substance may be present in an amount of about 0.01% to about 9% by weight of the consumable. In some embodiments, the active substance may be present in an amount of about 0.01% to about 8% by weight of the consumable. In some embodiments, the active substance may be present in an amount of about 0.01% to about 7% by weight of the consumable. In some embodiments, the active substance may be present in an amount of about 0.01% to about 6% by weight of the consumable. In some embodiments, the active substance may be present in an amount of about 0.01% to about 5% by weight of the consumable. In some embodiments, the active substance may be present in an amount of about 0.01% to about 4% by weight of the consumable. In some embodiments, the active substance may be present in an amount of about 0.01% to about 3% by weight of the consumable. In some embodiments, the active substance may be present in an amount of about 0.01% to about 2% by weight of the consumable. In some embodiments, the active substance may be present in an amount of about 0.01% to about 1% by weight of the consumable.

Throughout this disclosure, the term “by weight of the consumable” means the total weight of the consumable.

In other embodiments, for example when the active substance is nicotine or a salt thereof, the active subject may be present in an amount of no greater than 6% by weight of the consumable. Such as no greater than 5, 4, 3, 2, or 1% by weight. Each of these ranges may have a lower limit of 0.1, 0.2, 0.3, 0.4, 0.5, 0.6, 0.7 or 0.8% by weight.

In one embodiment, nicotine is present in an amount of from about 0.4 to about 6 wt % based on the total weight of the consumable. In one embodiment, nicotine is present in an amount of from about 0.8 to about 6 wt % based on the total weight of the consumable. In one embodiment, nicotine is present in an amount of from about 1 to about 6 wt % based on the total weight of the consumable. In one embodiment, nicotine is present in an amount of from about 1.8 to about 6 wt % based on the total weight of the consumable.

In another embodiment, nicotine is present in an amount of no greater than about 3 wt % based on the total weight of the consumable. In one embodiment, nicotine is present in an amount of from about 0.4 to about 3 wt % based on the total weight of the consumable. In one embodiment, nicotine is present in an amount of from about 0.8 to about 3 wt % based on the total weight of the consumable. In one embodiment, nicotine is present in an amount of from about 1 to about 3 wt % based on the total weight of the consumable. In one embodiment, nicotine is present in an amount of from about 1.8 to about 3 wt % based on the total weight of the consumable.

In one embodiment, nicotine is present in an amount of less than about 1.9 wt % based on the total weight of the consumable. In one embodiment, nicotine is present in an amount of less than about 1.8 wt % based on the total weight of the consumable. In one embodiment, nicotine is present in an amount of from about 0.4 to less than about 1.9 wt % based on the total weight of the consumable. In one embodiment, nicotine is present in an amount of from about 0.4 to less than about 1.8 wt % based on the total weight of the consumable. In one embodiment, nicotine is present in an amount of from about 0.5 to less than about 1.9 wt % based on the total weight of the consumable. In one embodiment, nicotine is present in an amount of from about 0.5 to less than about 1.8 wt % based on the total weight of the consumable. In one embodiment, nicotine is present in an amount of from about 0.8 to less than about 1.9 wt % based on the total weight of the consumable. In one embodiment, nicotine is present in an amount of from about 0.8 to less than about 1.8 wt % based on the total weight of the consumable. In one embodiment, nicotine is present in an amount of from about 1 to less than about 1.9 wt % based on the total weight of the consumable. In one embodiment, nicotine is present in an amount of from about 1 to less than about 1.8 wt % based on the total weight of the consumable.

In one embodiment, the consumable does not contain nicotine.

In various embodiments of the present disclosure, the consumable comprises one or more aerosol-former materials. The aerosol-former material is a material that is capable of generating aerosol, for example, when heated, irradiated or energized in any other way. The term “aerosol-generating material” may be used interchangeably with “aerosol-former material”. Aerosol-former material may, for example, be in the form of a solid, liquid or gel, which may or may not contain an active substance or ingredient as defined hereinabove. The aerosol-former material may further comprise one or more additional flavouring agents, and optionally one or more other functional material, all as defined herein.

In some embodiments, the aerosol-former material may comprise an “amorphous solid”, which may alternatively be referred to as a “monolithic solid” (i.e. non-fibrous). In some embodiments, the amorphous solid may be a dried gel. The amorphous solid is a solid material that may retain some fluid, such as liquid, within it. In some embodiments, the aerosol-former material may for example comprise from about 50 wt %, 60 wt %, or 70 wt % of amorphous solid, to about 90 wt %, 95 wt %, or 100 wt % of amorphous solid.

In some embodiments, the aerosol-former material may comprise one or more of glycerol, propylene glycol, diethylene glycol, triethylene glycol, tetraethylene glycol, 1,3-butylene glycol, erythritol, meso-erythritol, ethyl vanillate, ethyl laurate, a diethyl suberate, triethyl citrate, triethylene glycol diacetate, triacetin, a diacetin mixture, benzyl benzoate, benzyl phenyl acetate, tributyrin, lauryl acetate, lauric acid, myristic acid, and propylene carbonate. In some embodiments, the aerosol-former material comprises propylene glycol. In some embodiments, the aerosol-former material comprises glycerol, for example propylene glycol, glycerol or a mixture thereof.

In one embodiment, the aerosol-former material is present in an amount of from 10% w/w to 95% w/w based on the total weight of the consumable. In one embodiment, aerosol-former material is present in an amount of from 20% w/w to 95% w/w based on the total weight of the consumable. In one embodiment, aerosol-former material is present in an amount of from 30% w/w to 95% w/w based on the total weight of the consumable. In one embodiment, aerosol-former material is present in an amount of from 40% w/w to 95% w/w based on the total weight of the consumable.

In one embodiment, aerosol-former material is present in an amount of from 50% w/w to 90% w/w based on the total weight of the consumable. In one embodiment, aerosol-former material is present in an amount of from 50% w/w to 85% w/w based on the total weight of the consumable. In one embodiment, aerosol-former material is present in an amount of from 50% w/w to 80% w/w based on the total weight of the consumable. In one embodiment, aerosol-former material is present in an amount of from 50% w/w to 75% w/w based on the total weight of the consumable.

In one embodiment, aerosol-former material is present in an amount of at least 10% w/w based on the total weight of the consumable. In one embodiment, aerosol-former material is present in an amount of at least 20% w/w based on the total weight of the consumable. In one embodiment, aerosol-former material is present in an amount of at least 30% w/w based on the total weight of the consumable. In one embodiment, aerosol-former material is present in an amount of at least 40% w/w based on the total weight of the consumable. In one embodiment, aerosol-former material is present in an amount of at least 50% w/w based on the total weight of the consumable. In one embodiment, aerosol-former material is present in an amount of at least 55% w/w based on the total weight of the consumable. In one embodiment, aerosol-former material is present in an amount of at least 60% w/w based on the total weight of the consumable. In one embodiment, aerosol-former material is present in an amount of at least 65% w/w based on the total weight of the consumable. In one embodiment, aerosol-former material is present in an amount of at least 70% w/w based on the total weight of the consumable. In one embodiment, aerosol-former material is present in an amount of at least 75% w/w based on the total weight of the consumable. In one embodiment, aerosol-former material is present in an amount of at least 80% w/w based on the total weight of the consumable. In one embodiment, aerosol-former material is present in an amount of at least 85% w/w based on the total weight of the consumable. In one embodiment, aerosol-former material is present in an amount of at least 90% w/w based on the total weight of the consumable.

In some embodiments, the consumable comprises less than 12% w/w water. In some embodiments, the consumable comprises less than 11% w/w water. In some embodiments, consumable comprises less than 10% w/w water. In some embodiments, the consumable comprises less than 5% w/w water. In some embodiments, the consumable comprises less than 1% w/w water. In some embodiments, the consumable comprises less than 0.5% w/w water. In some embodiments, the consumable comprises substantially no water.

In one embodiment, both glycerol and propylene glycol are present as aerosol-former materials in the consumable. For example, glycerol and propylene glycol may be present in the consumable in the following amounts: 60 to 90% w/w propylene glycol; and 40 to 10% w/w glycerol, based on the total weight of glycerol and propylene glycol present in the material. In one embodiment, glycerol and propylene glycol are present in the consumable in the following amounts: 70 to 80% w/w propylene glycol, and 30 to 20% w/w glycerol, based on the total weight of glycerol and propylene glycol present in the consumable.

In one embodiment, the consumable is a liquid at about 25° C.

In various embodiments of the present disclosure, the composition optionally includes one or more sensate compounds but is free from any other additional flavourants or flavouring agents. The composition does not, for example, include a flavouring agent to provide the additional aromas mentioned above: citrus, fruity, herbal, floral, creamy, roasted, nutty or a mixture thereof. Such aromas are provided by the flavoured composition obtained from the biomimetic approach discussed herein.

In other embodiments of the present disclosure the consumable comprises one or more additional flavours or flavouring agents which may include naturally occurring flavour materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavour enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gas.

In some embodiments, the additional flavour comprises menthol, spearmint and/or peppermint. In some embodiments, the additional flavour comprises eugenol. In some embodiments, the additional flavour comprises flavour components extracted from tobacco. In some embodiments, the additional flavour comprises flavour components extracted from cannabis.

In some embodiments, the total amount of the additional flavour present in the consumable is up to about 10 w/w %. In some embodiments, the total amount of the additional flavour present in the consumable is up to about 9 w/w %. In some embodiments, the total amount of the additional flavour present in the consumable is up to about 8 w/w %. In some embodiments, the total amount of the additional flavour present in the consumable is up to about 7 w/w %. In some embodiments, the total amount of the additional flavour present in the consumable is up to about 6 w/w %. In some embodiments, the total amount of the additional flavour present in the consumable is up to about 5 w/w %. In some embodiments, the total amount of the additional flavour present in the consumable is up to about 4 w/w %. In some embodiments, the total amount of the additional flavour present in the consumable is up to about 3 w/w %. In some embodiments, the total amount of the additional flavour present in the consumable is up to about 2 w/w %. In some embodiments, the total amount of the additional flavour present in the consumable is up to about 1 w/w %.

In various embodiments of the present disclosure, the composition or the consumable further comprises one or more sensate compounds. A sensate compound is one which is intended to achieve a somatosensorial sensation usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether, and a suitable cooling agent may be, but not limited to eugenol, WS-3, WS-23. The composition may, for example, include a sensate compound selected from the group consisting of: WS-3, WS-23, carvacrol, eugenol, 1,8-cineole, and 4-methoxycinnamaldehyde.

In some embodiments, the total amount of the sensate compound present in the composition is up to about 10 w/w %. In some embodiments, the total amount of the sensate compound present in the composition is up to about 9 w/w %. In some embodiments, the total amount of the sensate compound present in the composition is up to about 8 w/w %. In some embodiments, the total amount of the sensate compound present in the composition is up to about 7 w/w %. In some embodiments, the total amount of the sensate compound present in the composition is up to about 6 w/w %. In some embodiments, the total amount of the sensate compound present in the composition is up to about 5 w/w %. In some embodiments, the total amount of the sensate compound present in the composition is up to about 4 w/w %. In some embodiments, the total amount of the sensate compound present in the composition is up to about 3 w/w %. In some embodiments, the total amount of the sensate compound present in the composition is up to about 2 w/w %. In some embodiments, the total amount of the sensate compound present in the composition is up to about 1 w/w %.

In various embodiments of the present disclosure the consumable comprises one or more substrate materials. By the term “substrate material” is meant a substance or material which supports or otherwise carries the flavoured composition. The substrate material may be a solid, liquid or gel. In various embodiments of the present disclosure, the substrate material is a solid or a gel. The substrate material may for example, be a fibrous material, such as a material selected from the group consisting of paper, tobacco, non-tobacco plant material (e.g. cellulose) or combinations thereof. A cellulose substrate may, for example, comprise cellulose acetate or derivatives thereof including a cellulose acetate filter in a smoking article.

The substrate may comprise a non-tobacco plant material including cereal grains (e.g., maize, oat, barley, rye, buckwheat, and the like), sugar beet (e.g., FIBREX® brand filler available from International Fiber Corporation), bran fiber, and mixtures thereof. Non-limiting examples of derivatives of non-tobacco plant material include starches (e.g., from potato, wheat, rice, corn), natural cellulose, and modified cellulosic materials. In some embodiments, the non-tobacco plant material substrate comprises a starch, a cellulose material, or both. A cellulose material may, for example, be used to enhance certain organoleptic properties such as texture and mouthfeel, enhancing cohesiveness or compressibility of the consumable for oral use.

In some embodiments, the cellulose material may be microcrystalline cellulose (mcc). The mcc may be synthetic or semi-synthetic, or it may be obtained entirely from natural celluloses. The cellulose derivative may, for example, be present from about 10 to 80% by weight of the consumable. In some embodiments, the cellulose substrate, e.g. the cellulose derivative substrate, is present from about 10 to 70% by weight of the consumable. When a cellulose substrate is present, the consumable may comprise no more than about 10% by weight of a tobacco material, excluding any nicotine present, based on the total weight of the consumable. In various embodiments the consumable comprising a non-tobacco plant material substrate (e.g. cellulose) includes no more than about 5% by weight of a tobacco material, excluding any nicotine present, based on the total weight of the consumable.

The tobacco material, whether included in addition to the non-plant material substrate or used as a substrate itself, may be prepared from any type or form of tobacco. The present disclosure is not limited in this respect. Generally, the tobacco material is obtained from a harvested plant of the Nicotiana species. Example Nicotiana species include N. tabacum, N. rustica, N. alata, N. arentsii, N. excelsior, N. forgetiana, N. glauca, N. glutinosa, N. gossei, N. kawakamii, N. knightiana, N. langsdorffi, N. otophora, N. setchelli, N. sylvestris, N. tomentosa, N. tomentosiformis, N. undulata, N. x sanderae, N. africana, N. amplexicaulis, N. benavidesii, N. bonariensis, N. debneyi, N. longiflora, N. maritina, N. megalosiphon, N. occidentalis, N. paniculata, N. plumbaginifolia, N. raimondii, N. rosulata, N. simulans, N. stocktonii, N. suaveolens, N. umbratica, N. velutina, N. wigandioides, N. acaulis, N. acuminata, N. attenuata, N. benthamiana, N. cavicola, N. clevelandii, N. cordifolia, N. corymbosa, N. fragrans, N. goodspeedii, N. linearis, N. miersii, N. nudicaulis, N. obtusifolia, N. occidentalis subsp. Hersperis, N. pauciflora, N. petunioides, N. quadrivalvis, N. repanda, N. rotundifolia, N. solanifolia, and N. spegazzinii.

Various representative other types of plants from the Nicotiana species are set forth in Goodspeed, The Genus Nicotiana, (Chonica Botanica) (1954); U.S. Pat. No. 4,660,577 to Sensabaugh, Jr. et al.; 5,387,416 to White et al., U.S. Pat. No. 7,025,066 to Lawson et al.; U.S. Pat. No. 7,798,153 to Lawrence, Jr. and U.S. Pat. No. 8,186,360 to Marshall et al.; each of which is incorporated herein by reference. Descriptions of various types of tobaccos, growing practices and harvesting practices are set forth in Tobacco Production, Chemistry and Technology, Davis et al. (Eds.) (1999), which is incorporated herein by reference.

Various parts or portions of the plant of the Nicotiana species can be included within a tobacco material as disclosed herein. For example, virtually all of the plant (e.g., the whole plant) can be harvested, and employed as such. Alternatively, various parts or pieces of the plant can be harvested or separated for further use after harvest. For example, the flower, leaves, stem, stalk, roots, seeds, and various combinations thereof, can be isolated for further use or treatment.

When nicotine is present in the consumable, it may be added or may be inherently present if the substrate material is a tobacco substrate material. In one embodiment the substrate material includes at least one tobacco substrate material. The tobacco substrate material may be solid, liquid or gel. In one embodiment, the tobacco substrate material is solid. The identity of the tobacco is not limited; it can be any type or grade of tobacco and includes any part, such as for example, the leaves or stems of any member of the genus Nicotiana and reconstituted materials thereof. In one embodiment, the tobacco is from the species Nicotiana tabacum. The tobacco substrate material may be from one variety of tobacco or from more than one variety of tobacco. As is known in the art, the latter can be referred to as a blend. Examples of tobacco varieties which may be used include, but are not limited to, Virginia, Burley, Oriental and Rustica tobaccos.

In one embodiment the tobacco substrate material is a pH-treated tobacco material; pH treatment of tobacco is well known in the art. In general, pH treatment raises the pH of the tobacco material from an acidic pH to an alkaline pH. The tobacco substrate material, including when the tobacco substrate material is a pH-treated tobacco material, can be in any suitable form. In one embodiment, the tobacco substrate material is in the form of particles, beads, granules or the like. The shape and/or size of such particles, beads or granules is not limited in the context of the present disclosure. The skilled person will be aware of suitable sizes and shapes and the methods by which such sizes and shapes can be achieved.

In another embodiment, the substrate material includes a non-tobacco substrate material.

The one or more other functional materials may depend on the type of substance delivery system in which the composition is to be used and may comprise one or more of pH regulators, humectants, colouring agents, preservatives, binders, fillers, stabilizers, and/or antioxidants. In particular, the pH regulator may include one or more acids selected from organic or inorganic acids. An example of an inorganic acid is phosphoric acid. The organic acid may include a carboxylic acid. The carboxylic acid may be any suitable carboxylic acid. In one embodiment, the acid is a mono-carboxylic acid. In one embodiment, the acid may be selected from the group consisting of acetic acid, lactic acid, formic acid, citric acid, benzoic acid, pyruvic acid, levulinic acid, succinic acid, tartaric acid, oleic acid, sorbic acid, propionic acid, phenylacetic acid, and mixtures thereof.

In certain embodiments, one or more humectants may be employed in the consumable, for example, with a tobacco or non-plant material substrate. Examples of humectants include, but are not limited to, glycerin, propylene glycol, and the like. Where included, the humectant is typically provided in an amount sufficient to provide desired moisture attributes to the mixture. Further, in some instances, the humectant may impart desirable flow characteristics to the mixture for depositing in a mold. When present, a humectant will typically make up about 5% or less of the weight of the mixture (e.g., from about 0.5 to about 5%). When present, a representative amount of humectant is about 0.1% to about 1% by weight, or about 1% to about 5% by weight, based on the total weight of the mixture.

### Container

The present disclosure further relates to a container or package containing the consumable as defined herein. The container or package may independently comprise one or more additional components as defined above. Specifically the container may comprise active constituents, one or more additional flavours, one or more sensate compounds, one or more aerosol-former materials, and/or one or more other functional materials.

In various embodiments of the present disclosure, the container or package is a pouched oral product comprising a saliva permeable pouch and the consumable defined herein. The consumable may be incorporated within the pouch. In such embodiments, the consumable comprises a substrate material which is a non-tobacco plant material, such as a cellulose material or a tobacco substrate material. In other embodiments of the present disclosure, the container or package is a cartomiser for an aerosol provision system and the consumable includes an aerosol-former material.

### Substance Delivery System

The present disclosure provides a substance delivery system comprising the consumable defined herein or the container or package defined herein. The substance delivery system can be implemented as a combustible aerosol provision system, a non-combustible aerosol provision system or an aerosol-free delivery system. In more detail, these systems are as follows:
combustible aerosol provision systems, such as cigarettes, cigarillos, cigars, and tobacco for pipes or for roll-your-own or for make-your-own cigarettes (whether based on tobacco, tobacco derivatives, expanded tobacco, reconstituted tobacco, tobacco substitutes or other smokeable material);
non-combustible aerosol provision systems that release compounds from an aerosol-generating material without combusting the aerosol-generating material, such as electronic cigarettes, tobacco heating products, and hybrid systems to generate aerosol using a combination of aerosol-generating materials; and
aerosol-free delivery systems that deliver the at least one substance to a user orally, nasally, transdermally or in another way without forming an aerosol, including but not limited to, lozenges, gums, patches, articles comprising inhalable powders, and oral products such as oral tobacco which includes snus or moist snuff, wherein the at least one substance may or may not comprise nicotine.

According to the present disclosure, a “combustible” aerosol provision system is one where a constituent aerosol-generating material of the aerosol provision system (or component thereof) is combusted or burned during use in order to facilitate delivery of at least one substance to a user. In some embodiments, the delivery system is a combustible aerosol provision system, such as a system selected from the group consisting of a cigarette, a cigarillo and a cigar. In some embodiments, the disclosure relates to a component for use in a combustible aerosol provision system, such as a filter, a filter rod, a filter segment, a tobacco rod, a spill, an aerosol-modifying agent release component such as a capsule, a thread, or a bead, or a paper such as a plug wrap, a tipping paper or a cigarette paper.

In various embodiments of the present disclosure, the substance delivery system is a combustible aerosol provision system. In various embodiments of the present disclosure the substance delivery system is a component of a combustible aerosol provision system such as a filter, a filter rod, a filter segment, a tobacco rod, a spill, an aerosol-modifying agent release component such as a capsule, a thread, or a bead, or a paper such as a plug wrap, a tipping paper or a cigarette paper.

According to the present disclosure, a “non-combustible” aerosol provision system is one where a constituent aerosol-generating material of the aerosol provision system (or component thereof) is not combusted or burned in order to facilitate delivery of at least one substance to a user. In some embodiments, the delivery system is a non-combustible aerosol provision system, such as a powered non-combustible aerosol provision system. In some embodiments, the non-combustible aerosol provision system is an electronic cigarette, also known as a vaping device or electronic nicotine delivery system (END), although it is noted that the presence of nicotine in the aerosol-generating material is not a requirement. In some embodiments, the non-combustible aerosol provision system is an aerosol-generating material heating system, also known as a heat-not-burn system. An example of such a system is a tobacco heating system.

In some embodiments, the non-combustible aerosol provision system is a hybrid system to generate aerosol using a combination of aerosol-generating materials, one or a plurality of which may be heated. Each of the aerosol-generating materials may be, for example, in the form of a solid, liquid or gel and may or may not contain nicotine. In some embodiments, the hybrid system comprises a liquid or gel aerosol-generating material and a solid aerosol-generating material. The solid aerosol-generating material may comprise, for example, tobacco or a non-tobacco product.

In various embodiments of the present disclosure, the substance delivery system is a non-combustible aerosol provision system. Typically, the non-combustible aerosol provision system may comprise a non-combustible aerosol provision device and a consumable for use with the non-combustible aerosol provision device. In some embodiments, the disclosure relates to consumables comprising aerosol-former material and the mint-flavoured composition, and configured to be used with non-combustible aerosol provision devices. These consumables are sometimes referred to as articles throughout the disclosure.

In some embodiments, the non-combustible aerosol provision system, such as a non-combustible aerosol provision device thereof, may comprise a power source and a controller.

The power source may, for example, be an electric power source or an exothermic power source. In some embodiments, the exothermic power source comprises a carbon substrate, which may be energised to distribute power in the form of heat to an aerosol-generating material or to a heat transfer material in proximity to the exothermic power source.

In some embodiments, the non-combustible aerosol provision system may comprise an area for receiving the consumable, an aerosol generator, an aerosol generation area, a housing, a mouthpiece, a filter and/or an aerosol-modifying agent. In some embodiments, the consumable for use with the non-combustible aerosol provision device may comprise aerosol-generating material, an aerosol-generating material storage area, an aerosol-generating material transfer component, an aerosol generator, an aerosol generation area, a housing, a wrapper, a filter, a mouthpiece, and/or an aerosol-modifying agent.

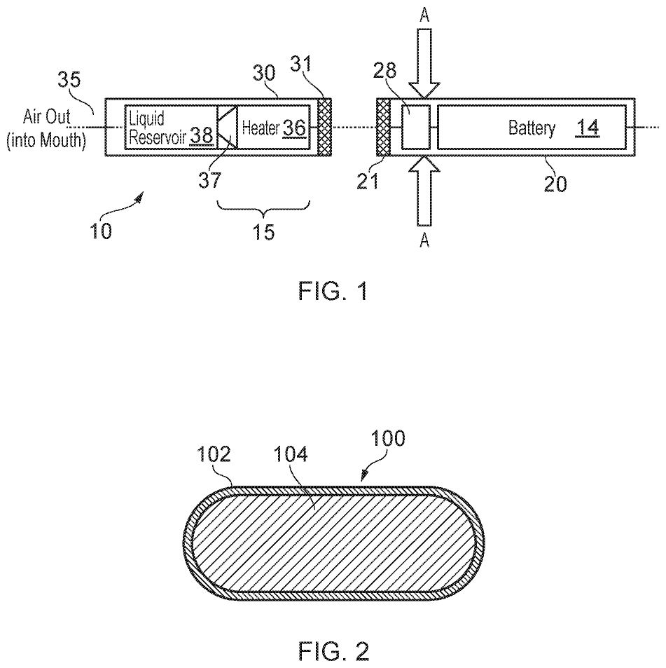

FIG. 1 is a highly schematic diagram (not to scale) of an example aerosol provision system, such as an e-cigarette 10, to which embodiments are applicable. The e-cigarette has a generally cylindrical shape, extending along a longitudinal axis indicated by a dashed line (although aspects of the present disclosure are applicable to e-cigarettes configured in other shapes and arrangements), and comprises two main components, namely an aerosol provision device 20 and an article 30.

The article 30 includes a store for aerosolisable material (source liquid) 38 containing an aerosolisable material (source liquid) from which an aerosol is to be generated. This material may correspond to the consumable according to the present disclosure or the consumable may be included in a separate compartment through which heated air passes through (not shown in FIG. 1). The article 30 further comprises an aerosol generating component (heating element or heater) 36 for heating aerosolisable material to generate the aerosol. A transport element or wicking element or wick 37 is provided to deliver aerosolisable material from the store 38 to the heating element 36. A part or parts of the wick 37 are in fluid communication with aerosolisable material in the store 38 and by a wicking or capillary action aerosolisable material is drawn along or through the wick 37 to a part or parts of the wick 37 which are in contact with the heater 36.

Vaporization of the aerosolisable material occurs at the interface between the wick 37 and the heater 36 by the provision of heat energy to the aerosolisable material to cause evaporation, thus generating the aerosol. The aerosolisable material, the wick 37 and the heater 36 may be collectively referred to as an aerosol or vapour source. The wick 37 and the heater 36 may be collectively referred to as a vaporizer or an atomiser 15. Typically, a single wick will be present, but it is envisaged that more than one wick could be present, for example, two, three, four or five wicks. The wick may be formed a sintered material. The sintered material may comprise sintered ceramic, sintered metal fibers/powders, or a combination of the two. The (or at least one of/all of the) sintered wick(s) may have deposited thereon/embedded therein an electrically resistive heater. Such a heater may be formed from heat conducting alloys such as NiCr alloys. Alternatively, the sintered material may have such electrical properties such that when a current is passed there through, it is heated. Thus, the aerosol-generating component and the wick may be considered to be integrated. In some embodiments, the aerosol-generating component and the wick are formed from the same material and form a single component.

The article 30 further includes a mouthpiece 35 having an opening through which a user may inhale the aerosol generated by the vaporizer 15. The aerosol for inhalation may be described as an aerosol stream or inhalable airstream.

The aerosol delivery device 20 includes a power source (a re-chargeable cell or battery 14, referred to herein after as a battery) to provide power for the e-cigarette 10, and a controller (printed circuit board (PCB)) 28 and/or other electronics for generally controlling the e-cigarette 10. The aerosol delivery device can therefore also be considered as a battery section, or a control unit or section. During operation of the device, the controller will determine that a user has initiated a request for the generation of an aerosol. This could be done via a button on the device which sends a signal to the controller that the aerosol generator should be powered.

Alternatively, a sensor located in or proximal to the airflow pathway could detect airflow through the airflow pathway and convey this detection to the controller. A sensor may also be present in addition to the presence of a button, as the sensor may be used to determine certain usage characteristics, such as airflow, timing of aerosol generation etc. For example, in use, when the heater 36 receives power from the battery 14, as controlled by the circuit board 28 possibly in response to pressure changes detected by an air pressure sensor (not shown), the heater 36 vaporizes aerosolisable material delivered by the wick 37 to generate the aerosol, and this aerosol stream is then inhaled by a user through the opening in the mouthpiece 35. The aerosol is carried from the aerosol source to the mouthpiece 35 along an air channel (not shown in FIG. 1) that connects the aerosol source to the mouthpiece opening as a user inhales on the mouthpiece.

In this particular example, the device 20 and article 30 are detachable from one another by separation in a direction parallel to the longitudinal axis, as shown in FIG. 1, but are joined together when the system 10 is in use by cooperating engagement elements 21, 31 (for example, a screw, magnetic or bayonet fitting) to provide mechanical and electrical connectivity between the device 20 and the article 30, in particular connecting the heater 36 to the battery 14. The battery may be charged as is known to one skilled in the art.

In some embodiments, the article comprises/forms a sealed container. For example, the sealed container may be hermetically sealed. The hermetically sealed container may comprise a blister pack with one or more hermetically sealed compartments for storage of one or more articles comprising the composition described herein.

In some embodiments, the substance delivery system is an aerosol-free delivery system that delivers at least one substance to a user orally, nasally, transdermally or in another way without forming an aerosol, including but not limited to, lozenges, gums, patches, articles comprising inhalable powders, and oral products such as oral tobacco which includes snus or moist snuff, wherein the at least one substance may or may not comprise nicotine.

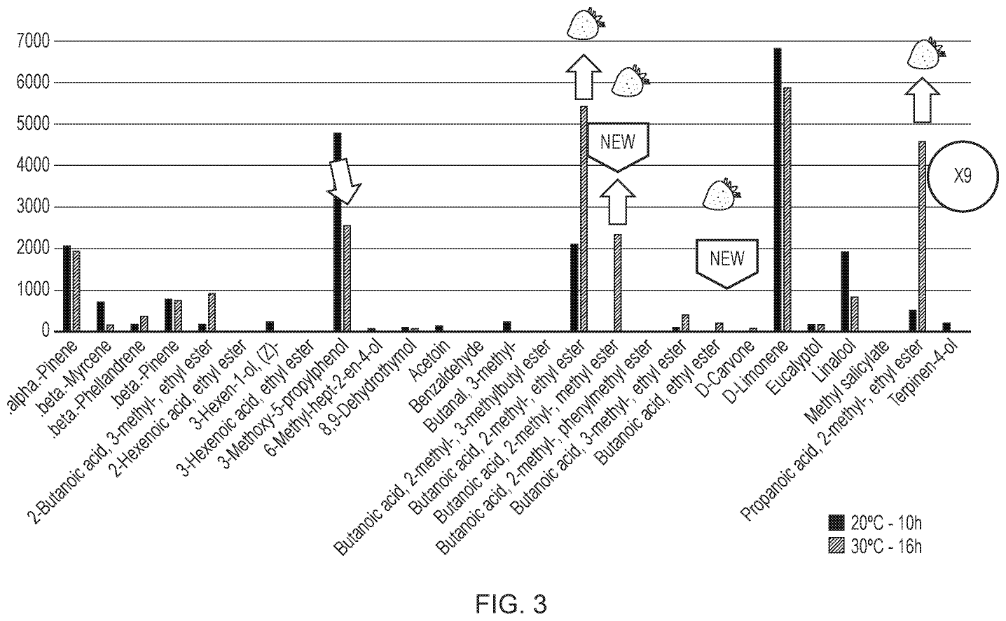

In various embodiments of the present disclosure, the substance delivery system is an aerosol-free delivery system. The aerosol-free delivery system may, for example, be a smokeless tobacco composition comprising the consumable where at least one tobacco material acts as a substrate for the mint-flavoured composition, or an oral product where the oral product is in the form of a pouch comprising the consumable. FIG. 2 is a cross-sectional view of a pouched product 100, taken across the width of the product, showing an outer pouch 102 filled with a consumable 104 of the present disclosure.

The present disclosure will now be described with reference to the following non-limiting examples.

### Examples

### Example 1: Enhancing the Orthonasal Profile of Strawberry Mint

A cultivar of strawberry mint plants was obtained and following determination of an initial threshold orthonasal profile according to the present disclosure, subjected to four test growth conditions as follows; the growth duration in each test was 4 weeks:
Test 1: 10 hours light exposure—20° C. day—15° C. night
Test 2: 10 hours light exposure—30° C. day—25° C. night
Test 3: 16 hours light exposure—20° C. day—15° C. night
Test 4: 16 hours light exposure—30° C. day—25° C. night

FIG. 3 compares the threshold orthonasal profile of a plant subjected to Test 1 with the threshold orthonasal profile of a plant subjected to Test 4. It can be seen that the increased temperature (30° C.) and the increased light exposure time (16 hours) led to a decrease in 3-methoxy-5-propylphenol, and an increase in butanoic acid, 2-methyl-, ethyl ester (otherwise known as ethyl 2-methylbutanoate (CAS 7452-79-1)), this compound is associated with a fruity, estry and berry odour with fresh tropical nuances, see for example http://www.thegoodscentscompany.com/data/rw1004871.html).

An increase was also seen for propanoic acid, 2-methyl-, ethyl ester (otherwise known as ethyl 2-methylpropanoate (CAS 97-62-1)), associated with a sweet, ethereal and fruity odour with pungent, alcoholic, fusel and rummy nuances, see http://www.thegoodscentscompany.com/data/rw1000751.html, as well as new peaks for butanoic acid, 2-methyl-, methyl ester (methyl 2-hydroxy-2-methyl butanoate (CAS 868-57-5); http://www.thegoodscentscompany.com/data/rw1624291.html) and butanoic acid, ethyl ester (ethyl butanoate (CAS 105-54-1) http://www.thegoodscentscompany.com/data/rw1004792.html).

These esters are associated with the strawberry aroma and thus result in an enhanced strawberry character. Specifically the strawberry mint without light treatment had a “wild strawberry profile” (methyl salicylate) with more animal notes. Through the light/temperature treatment, the number and concentration of the esters doubled, increasing the strawberry character to 76% of the total aroma profile.

### Example 2: Reconstructing the Orthonasal Profile of Enhanced Strawberry Mint

The enhanced strawberry mint plant of Example 1 was characterised using headspace technology coupled with GC-MS and GC-O to determine its threshold orthonasal profile. This profile consisted of the following compounds and amounts:

| Aroma type | Compound | Amount (ppm) |
|---|---|---|
| Fruity | Ethyl 2-methylbutanoate | 5.4 |
| | Ethyl 2-methylpropanaote | 4.5 |
| | Methyl 2-methylbutanoate | 2.4 |
| | 3-methyl-1-butanol | 1 |
| | Ethyl 3-methylbutanoate | 0.15 |
| | Ethyl butanoate | 0.32 |
| | Methyl hexanoate | 0.04 |
| Creamy | δ-decenolactone | 0.144 |
| | γ-decalatone | 0.05 |
| Roasted | 3-methylbutanal | 1.2 |
| Floral | Linalool | 2.3 |
| | Phenylacetaldehyde | |
| Berry | 3-mercapto-3- | 0.00008 ppm |
| | methylbutylformiate | |
| Green | (Z)-3-hexen-1-ol | 0.05 |
| Mint | Piperitenone | 7.2 |
| | D-carvone | 0.05 |
| | Alpha-pinene | 2 |
| | Beta-pinene | 1.1 |
| | Beta-phellandrene | 0.37 |

This orthonasal profile was reconstructed using natural or synthetic sources of the identified compounds.

### Example 3: Enhancing the Orthonasal Profile of Apple Mint

A cultivar of apple mint (Mentha suaveolens) plants was obtained and following determination of an initial threshold orthonasal profile according to the present disclosure, subjected to the same four test growth conditions as Example 1; the growth duration in each test was either 2 weeks or 4 weeks.

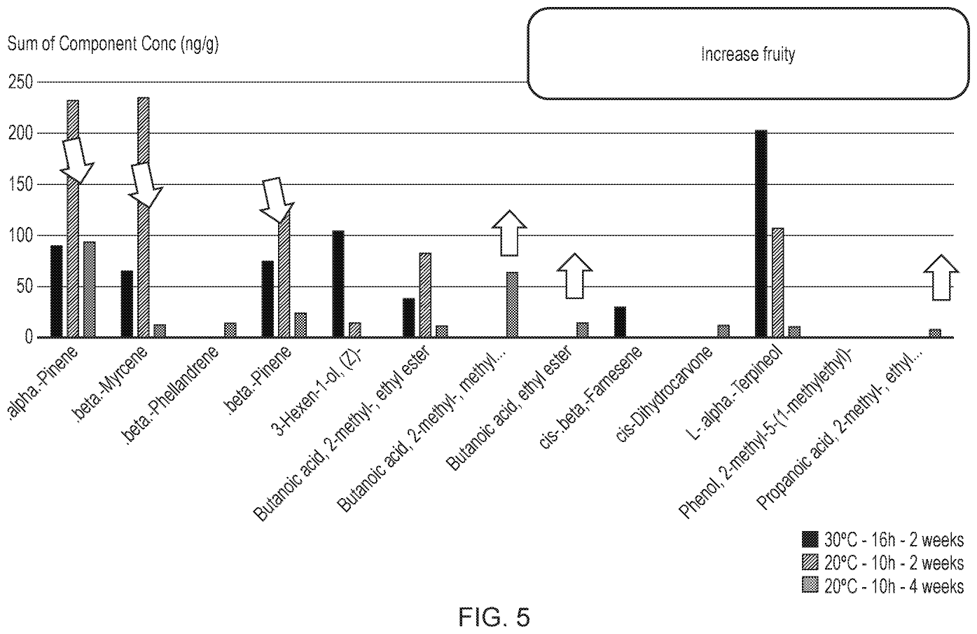

FIG. 4 compares the threshold orthonasal profile of a plant subjected to Test 4 for 2 weeks (30° C.-16 h-2 weeks) with a plant subjected to Test 1 for 2 weeks (20° C.-10 h-2 weeks) or 4 weeks (20° C.-10 h-4 weeks). It can be seen how the compounds associated with a mint aroma, namely D-carvone, D-limonene and eucalyptol were reduced as the light exposure time and temperature decreased. Reduction in the concentration of mint aroma compounds is also evident from FIG. 5: alpha-pinene, beta-myrcene and beta-pinene.

FIG. 5 compares the threshold orthonasal profile of the same plants as FIG. 4 and shows how Test 1 for 4 weeks (10 h-20° C.) increased the esters associated with the fruity aroma. Notably butanoic acid, 2-methyl-, methyl ester (methyl 2-hydroxy-2-methyl butanoate (CAS 868-57-5); (http://www.thegoodscentscompany.com/data/rw1624291.html:
butanoic acid, ethyl ester (ethyl butanoate (CAS 105-54-1) http://www.thegoodscentscompany.com/data/rw1004792.html); and propanoic acid, 2-methyl-, ethyl ester (otherwise known as ethyl 2-methylpropanoate (CAS 97-62-1)), associated with a sweet, ethereal and fruity odour with pungent, alcoholic, fusel and rummy nuances http://www.thegoodscentscompany.com/data/rw1000751.html.

### Example 4: Reconstructing the Orthonasal Profile of Enhanced Apple Mint

From FIGS. 4 and 5, the threshold orthonasal profile of the enhanced apple mint after Test 1 for 4 weeks can be defined as follows:

| Aroma type | Compound | Amount (ppm) |
|---|---|---|
| Fruity | Ethyl 2-methylbutanoate | 0.19 |
| | Methyl 2-methylbutanoate | 0.05 |
| | Ethyl 3-methylbutanoate | 0.04 |
| | Ethyl butanoate | 0.014 |
| | Ethyl isobutyrate | 0.0065 |
| Floral | Cis-jasmone | 0.08 |
| Green | (Z)-3-hexen-1-ol | 0.02 |
| Creamy/Nutty | δ-octalactone | 0.01 |
| Mint | D-carvone | 8.8 |
| | Menthone | 0.006 |
| | Alpha-pinene | 0.54 |
| | 1,8-cineole | 1.7 |
| | Limonene | 7.6 |
| | Beta-myrcene | 0.55 |

Apple mint with the growing conditions of Example 3 was about 60% ‘mint’ and about 40% ‘apple’. This orthonasal profile was reconstructed using natural or synthetic sources of the identified compounds.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc, other than those specifically described herein. In addition, this disclosure may include other inventions not presently claimed, but which may be claimed in future.

1. A method of enhancing the orthonasal profile of a Lamiaceae, Geraniaceae, Asteraceae, Myrtaceae or Ericaceae plant, the method comprising:
a. determining an initial orthonasal profile of the Lamiaceae, Geraniaceae, Asteraceae, Myrtaceae or Ericaceae plant, wherein the profile comprises a plurality of chemical compounds and their respective contents;
b. modifying the Lamiaceae, Geraniaceae, Asteraceae, Myrtaceae or Ericaceae plant through its growing conditions by controlling one or more parameters selected from light exposure time, temperature, relative humidity, elevation, light intensity, total sun radiation, growth duration or a combination thereof,
c. determining the orthonasal profile of the modified plant; and
d. optionally altering one or more parameters of the growing conditions in step (b) to increase and/or reduce the respective content(s) of one or more chemical compound(s) in the modified orthonasal profile relative to the initial orthonasal profile.

2. The method according to claim 1, wherein the method comprises:
d. altering one or more parameters of the growing conditions to increase the respective content(s) of one or more chemical compound(s) in the modified orthonasal profile relative to the initial orthonasal profile, wherein said chemical compound(s) is associated with an aroma other than mint.

3. The method according to claim 1, wherein the respective content(s) of the chemical compound(s) are odor activity values.

4. The method according to claim 1, wherein the determining an orthonasal profile comprises:
acquiring data from the plant, wherein the data characterizes a headspace of the plant;
identifying from the acquired data a plurality of chemical compounds and their respective contents within the headspace; and
comparing the plurality of chemical compounds and their respective contents with one or more libraries to characterize the plant by one or more aroma types.

5. The method according to claim 4, wherein the comparison with one or more libraries comprises a comparison with an aroma threshold library so as to select the compound(s) and respective content(s) associated with the orthonasal profile of the sample detectable by a human nose.

6. The method according to claim 4, wherein the method further comprises selecting one or more chemical compound(s) and their respective content(s) which are associated with at least one aroma other than mint, and altering one or more parameters of the growing conditions to increase the respective content(s) of the selected one or more chemical compound(s).

7. A method of preparing a mint-flavored composition for a substance delivery system, said mint-flavored composition having a predetermined orthonasal profile with at least one aroma other than mint, the method comprising:
a. identifying the predetermined orthonasal profile with at least one aroma other than mint, the orthonasal profile comprising a plurality of chemical compounds and their respective contents; and
b. providing each of the plurality of chemical compound(s) at their respective content(s) by including one or more natural or synthetic source(s) of said compound(s) in the composition.

8. The method according to claim 7, wherein the respective contents of the plurality of chemical compounds are odor activity values.

9. The method according to claim 7, wherein the predetermined orthonasal profile is identified by a method which comprises:
acquiring data from a Lamiaceae, Geraniaceae, Asteraceae, Myrtaceae or Ericaceae plant, wherein the data characterizes the headspace of the plant;
identifying from the acquired data a plurality of chemical compounds and their respective contents within the headspace;
comparing the plurality of chemical compounds and their respective contents with one or more libraries to characterize the plant by one or more aroma types; and
selecting one or more chemical compound(s) and respective content(s) which are associated with at least one aroma type other than mint.

10. The method according to claim 9, wherein the method further comprises selecting one or more chemical compound(s) and their respective content(s) which are associated with the mint aroma, and providing one or more of said mint aroma compound(s) at its respective content(s) by including a natural or synthetic source of said compound(s) in the composition.

11. The method according to claim 9, wherein the comparison with one or more libraries comprises a comparison with an aroma threshold library so as to select one or more chemical compound(s) and their respective content(s) associated with the orthonasal profile of the sample detectable by a human nose.

12. The method according to claim 9, wherein the natural source of the one or more chemical compound(s) is a botanical or constituent or extract thereof, such as an oil extracted from the botanical, provided that if all chemical compounds in the predetermined orthonasal profile are from a natural source, they are not from oils extracted from the botanical.

13. The method according to claim 12 wherein at least one of the chemical compound(s) is provided by an extract or constituent of a botanical, such as an extract or constituent from a Lamiaceae, Geraniaceae, Asteraceae, Myrtaceae or Ericaceae plant.

14. The method according to claim 9, wherein at least one of the chemical compound(s) is provided by a synthetic source.

15. The method according to claim 9, wherein the Lamiaceae, Geraniacae, Asteraceae, Myrtaceae or Ericaceae plant has an enhanced orthonasal profile obtained by the method of claim 1.

16. The method according to claim 1, wherein the one or chemical compound(s) are selected from the group consisting of esters, alcohols, lactones, terpenes, aldehydes or a combination thereof.

17. The method according to claim 2, wherein the aroma other than mint is selected from the group consisting of: citrus, fruity, floral, green, herbal, vegetable, creamy, roasted, nutty, woody, spicy, cheesy or a combination thereof.

18. The method according to claim 2, wherein the aroma other than mint is selected from the group consisting of: lemon, bergamot, orange, floral, grapefruit, lime, banana, pineapple, apple, pear, mango, basil, cinnamon, tropical, chocolate, honey, grass, clove, vanilla, strawberry, raspberry, ginger, lavender, coconut, melon, blackcurrant, grape, anise, rose, liquorice, root beer, coconut, grape, green, green pepper, hazelnut, marshmallow, peach, thyme, basil, oregano, bay leaf, or a combination thereof.

19. A mint-flavored composition obtainable from the method according to claim 7.

20. (canceled)

21. A consumable for use in a substance delivery system comprising the mint-flavored composition of claim 19, and at least one component selected from aerosol-former material, active substances, substrate materials, or a combination thereof.

22. (canceled)

23. (canceled)

24. (canceled)

25. (canceled)

26. (canceled)

27. (canceled)

28. A container or package containing the consumable of claim 21, such as a pouched oral product comprising a saliva permeable pouch.

29. A substance delivery system comprising the consumable of claim 21.

30. (canceled)

31. A Lamiaceae, Geraniaceae, Asteraceae, Myrtaceae or Ericaceae plant, which has been modified to increase and/or reduce the respective content of one or more chemical compound(s) of its orthonasal profile in comparison to an unmodified plant, wherein the modification is through the growing conditions of the plant, and the one or chemical compound(s) is associated with an aroma other than mint.

32. The method according to claim 7, wherein the one or chemical compound(s) are selected from the group consisting of esters, alcohols, lactones, terpenes, aldehydes or a combination thereof.

33. The method according to claim 9, wherein the aroma other than mint is selected from the group consisting of: citrus, fruity, floral, green, herbal, vegetable, creamy, roasted, nutty, woody, spicy, cheesy or a combination thereof.

34. The method according to claim 9, wherein the aroma other than mint is selected from the group consisting of: lemon, bergamot, orange, floral, grapefruit, lime, banana, pineapple, apple, pear, mango, basil, cinnamon, tropical, chocolate, honey, grass, clove, vanilla, strawberry, raspberry, ginger, lavender, coconut, melon, blackcurrant, grape, anise, rose, licorice, root beer, coconut, grape, green pepper, hazelnut, marshmallow, peach, thyme, basil, oregano, bay leaf, or a combination thereof.
